(19) United States
(12) Patent Application Publication
(10) Pub. No.: US 2018/0263547 A1
(43) Pub. Date: Sep. 20, 2018

(54) APPARATUS WITH A FORCE-SENSING INSTRUMENT FOR MAGNETIC RESONANCE IMAGING

(71) Applicant: Board of Regents of the University of Texas System, Austin, TX (US)

(72) Inventors: Floyd D. Settles, San Antonio, TX (US); Geoffrey D. Clarke, San Antonio, TX (US)

(21) Appl. No.: 15/848,551

(22) Filed: Dec. 20, 2017

Related U.S. Application Data
(60) Provisional application No. 62/436,931, filed on Dec. 20, 2016.
(60) Provisional application No. 62/436,948, filed on Dec. 20, 2016.
(60) Provisional application No. 62/436,951, filed on Dec. 20, 2016.

(51) Int. Cl. A61B 5/22; A63B 21/00; A61B 5/055; A63B 21/02; A63B 21/055

(52) U.S. Cl. CPC A61B 5/222; A63B 21/4033; A63B 21/0552; A63B 21/026; A61B 5/055

(57) ABSTRACT

An apparatus for measuring work performed by a subject in a magnetic resonance imaging device is provided which includes an actuating bar, an elastomer band positioned inside the actuating bar, a tower and roller bearing, and a pivot base supporting the actuating bar, the elastomer band positioned inside the actuating bar, and the tower and roller bearing. A force-sensing instrument for use in a magnetic resonance imaging device is provided, which includes a strain gauge cemented to a surface of an actuating device that is operable in a magnetic resonance imaging device. A surface coil holder having four degrees of freedom is provided. The surface coil holder is capable of being attached to a scanner table of an exercise apparatus. The surface coil holder includes a coil cradled in the coil holder and secured by hook and loop strips.

### RELATED APPLICATIONS

This application is a non-provisional application claiming priority to U.S. Prov. App. No. 62/436,931, filed Dec. 20, 2016, U.S. Prov. App. No. 62/436,948, filed Dec. 20, 2016, and U.S. Prov. App. No. 62/436,951, filed Dec. 20, 2016; each incorporated herein by reference in its entirety.

### GOVERNMENT LICENSE RIGHTS

This invention was made with government support under grant no. DK089012 awarded by the National Institutes of Health. The government has certain rights in the invention.

### TECHNICAL FIELD

The present disclosure generally relates to an exercise apparatus configured to function with a magnetic resonance scanner, a surface coil operable over certain muscles, and an adjustable coil holder. The present disclosure also provides specifically for an exercise apparatus (ergometer) compatible with a 3 Tesla Siemens Trip Magnetic Resonance scanner and a dual-tuned ¹H-³¹P transmit/receive (TX/RX) surface coil fixed over the vastus lateralis muscle, and an MRI-compatible adjustable coil holder. The present disclosure also provides a force-sensing instrument capable of being configured for use in a magnetic resonance imaging (MRI) device. The present disclosure also provides a method for quantitating mitochondrial dynamics in skeletal muscle of type 2 diabetes subjects using ³¹P magnetic resonance spectroscopy.

### BACKGROUND

Measurement of mitochondrial function is relevant to aging, diabetes and sports medicine. Phosphorus-31 magnetic resonance spectroscopy (³¹P-MRS) can be used as an in vivo biomarker to evaluate the rate of phosphocreatine (PCr) recovery following exercise as a noninvasive index of the rate of adenosine triphosphate (ATP) synthesis.

Type 2 diabetes mellitus is marked by impaired fatty acid oxidation, increased plasma fasting free fatty acid levels, and hyperglycemia. Since approximately 80% of total body glucose uptake occurs in skeletal muscle, the mechanisms by which type 2 diabetes mellitus alters muscle metabolism are of intense interest in diabetes research. In vivo concentrations of adenosine triphosphate and phosphocreatine, obtained using 1-hydrogen and 31-phosphorus magnetic resonance spectroscopy in the vastus lateralis muscle are less in type 2 diabetes mellitus subjects compared to normal glucose tolerant subjects.

A quadriceps muscle, the vastus lateralis muscle (VL m.) found in the outer thigh has been widely used as a source of muscle biopsies, especially those performed in diabetes research. Recent interest has grown in using VL m. as an in vivo target for quantifying the depletion levels and recovery times of PCr using ³¹P-MRS. During the normal exercise of muscle tissue, PCr is depleted and a rise in inorganic phosphate (Pi) occurs. Creatine (Cr) is a compound that acts as an energy reserve, providing a nitrogen binding site for a high energy phosphate group. Completion of the high energy phosphate bond produces phosphocreatine (PCr), which can later give up the energy of the phosphate bond at sites in the cell that require energy. Following periods of intense energy depletion during which ATP has been quickly consumed, this forward reaction is mediated through an isoform of the creatine kinase (CK) enzyme. Thus phosphocreatine acts as a buffer, temporarily supplying energy where needed until sufficient additional ATP can be produced in and transported from the mitochondria.

Located in the proton-rich environment of the inner mitochondrial wall, the mitochondrial ATP synthase enzyme builds ATP by joining ADP and Pi. The process is driven by a flux gradient of protons across the cell membrane due to electron transfer. The gradient goes from the positive (P) side in the intermembrane space to the negative (N) side in the mitochondrial matrix. The overall reaction is:

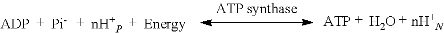

where ADP is adenosine diphosphate and nH⁺ represents proton flux. The reaction is catalyzed by the enzyme ATP synthase and, under favorable energetic circumstances, the reaction is reversible. The high energy phosphate bond is soon transferred to a Cr molecule through the reverse reaction of CK by an isomer located in the mitochondrial intermembrane space. The PCr then leaves the mitochondria and diffuses to the myofibrils and other sites of high energy usage, where it encounters the CK and ADP, undergoing the forward CK reaction and effectively delivering ATP for muscular contraction.

Following a period of exercise, during which the PCr is depleted and Pi is accumulated at the myofibrils, ATP will be produced as the creatine kinase will now restore the levels of PCr and Pi to their resting equilibrium values. The reversible PCr reaction equation is:

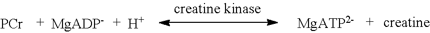

### SUMMARY

An apparatus for measuring work performed by a subject in a magnetic resonance imaging device is provided which includes an actuating bar, an elastomer band positioned inside the actuating bar, a tower and roller bearing, and a pivot base supporting the actuating bar, the elastomer band positioned inside the actuating bar, and the tower and roller bearing. A force-sensing instrument for use in a magnetic resonance imaging device is provided, which includes a strain gauge cemented to a surface of an actuating device that is operable in a magnetic resonance imaging device. A surface coil holder having four degrees of freedom is provided. The surface coil holder is capable of being attached to a scanner table of an exercise apparatus. The surface coil holder includes a coil cradled in the coil holder and secured by hook and loop strips.

The present disclosure was developed in an effort to address shortcomings in conventional MR-compatible ergometers by developing a repeatable MRS exercise protocol as a basis for performing ³¹P-MRS scans for research involving subjects with insulin resistance and type 2 diabetes mellitus (T2DM).

To achieve this goal, an exemplary embodiment of the present disclosure includes the design, development and prototyping of a safe, lightweight exercise apparatus, small enough to operate within the bore of the scanner and be compatible with the intense magnetic fields found inside an MRI system. The exercise apparatus provides resistance to the extension of the distal leg in order to exhaust the VL m. in a short period of time. To ensure the safety of the exercise, the use of massive weights is eliminated so that if the muscle gives out during the exercise period and the weight equivalent is dropped, both the subject and the scanner are protected from injury and/or damage.

In another exemplary embodiment, the apparatus was assembled and tested for functionality, comfort, safety and durability, and was able to accommodate a variety of subjects of different heights, weights, ages and physical abilities. Volunteers were fitted to the apparatus and a surface coil, configured as a unit, and performed the exercise protocol. Slice selective ³¹P-MRS data was collected on 11 subjects (5 female, 17-65 years old) with TR=3000 ms, NSA=2, BW=2200 Hz. The time for recovery of PCr values to their half-maximum (T-half) and/or the rate constant of recovery were used as indexes for the rate of ATP synthesis.

In another exemplary embodiment, the apparatus provides an indication of the relative amount of work being done by the subject during the exercise phase of the protocol. This was accomplished using strain gauge devices fused to the actuating bar where the force from the leg is applied. The work output data was captured by a digital acquisition module (DAQ) connected to the stain gauge circuitry and a Windows 7 computer. Software emulating a strip recorder was used to watch, in real-time, a subject's force of effort and the position of the actuating bar on the computer monitor. The software was configured to sample and collect data at a 50 Hz rate for 5 minutes and 20 seconds, which allows capturing a baseline 10 seconds before and after the 5 minutes exercise period. The data capture was written to an Excel compatible file for post processing.

In another exemplary embodiment, a method is provided to post process the exercise performance data to determine a relative quantity of work performed by each MRS study. Newtonian physics was applied to the data set to quantify a relative level of work imparted to the exercise apparatus.

In yet a further embodiment, the present disclosure provides an apparatus for measuring work performed by a subject in a magnetic resonance imaging device. The apparatus includes an actuating bar, an elastomer band positioned inside the actuating bar, and a tower and roller bearing. In such embodiments, a pivot base supports the actuating bar with the elastomer band positioned inside the actuating bar and the tower and roller bearing. In yet further embodiments, the apparatus may have at least four attach points to position, mount, and secure the ergometer to a secondary device. In some embodiments, the secondary device may be a magnetic resonance imaging device. In other embodiments, the apparatus further includes a magnetic resonance coil. In such embodiments, the magnetic resonance coil may be positioned close to the isocenter of the magnet of the magnetic resonance imaging device, and the vastus lateralis muscle of a subject and a primary axis of the coil are parallel to a magnetic field within a bore of the magnetic resonance imaging device.

In further embodiments, the material selected for the apparatus may include polyvinylchloride (PVC), common PVC cement, and non-magnetic stainless steel and nylon.

In further embodiments, the apparatus may be adjustable to accommodate subjects from 60 inches to 77 inches in height. In some embodiments, the actuating bar may have a single adjustment point to extend the length of the bar to match a subject's distal leg length. In yet further embodiments, the apparatus provides for the correct positioning of a leg of a subject. In certain embodiments, the apparatus isolates movement of the leg in the y-axis direction. In further embodiments, the actuating bar includes a pivot point with a slip bearing to withstand torque in two axes.

The present disclosure also provides a method for executing a magnetic resonance imaging protocol. The method includes (a) positioning and attaching an apparatus and a surface coil to a scanner couch of a magnetic resonance imaging device; and (b) positioning a subject on the apparatus and surface coil. In such embodiments, the apparatus includes a coil positioned close to the isocenter of a magnet of the magnetic resonance imaging device, an actuating bar, an elastomer band positioned inside the actuating bar, a tower and roller bearing, as well as a pivot base supporting the actuating bar, the elastomer band positioned inside the actuating bar, and the tower and roller bearing. In such embodiments, the apparatus may include at least four attach points to position, mount, and secure the apparatus to the magnetic resonance imaging device. The apparatus may also include a coil holder that includes the coil cradled in the coil holder and secured by a hook and loop strips.

In another exemplary embodiment, a force-sensing instrument for use in a magnetic resonance imaging device is provided. The force-sensing instrument includes a strain gauge cemented to a surface of an actuating device that is operable in a magnetic resonance imaging device. In further embodiments, the strain gauge is a copper foil electrical circuit, which is bonded to a thin plastic film. In yet further embodiments, the strain gauge includes two strain devices cemented to an actuating bar of the actuating device, and separated by 180±2 degrees. In yet further embodiments, the two strain devices form a Wheatstone bridge circuit along with two precision resistors, such that the two strain devices are positioned to sense strain in only one axis from a torque in a bar thereof. In another embodiment, the force-sensing instrument further includes a position indicator device. In some further embodiments, wherein the force-sensing instrument fits within an actuating bar of the actuating device.

Other embodiments of the present disclosure include a magnetic resonance imaging device that includes a force-sensing instrument. In such embodiments, the actuating device is adjustable to accommodate subjects from 60 inches to 77 inches in height.

Also provided in the present disclosure is a method of using the force-sensing instrumentation. The method includes attaching the force-sensing instrumentation to an ergometer which is attached to the scanner table of a magnetic resonance imaging device, adjusting the actuating device to accommodate a subject, operating the force-sensing instrumentation by the subject, delivering data obtained by the force-sensing instrumentation to a control room, performing a magnetic resonance imaging scan of the subject, delivering data obtained from the magnetic resonance imaging scan of the subject to a control room. In such embodiments, the step of operating the force-sensing instrumentation and the step of performing a magnetic resonance imaging scan overlap. Yet further embodiments provide for determining a relative amount of work being done by the subject while operating the force sensing instrumentation based on the data obtained by the force-sensing instrumentation and the data obtained by the magnetic resonance imaging scan. In further embodiments, the data obtained by the force-sensing instrumentation is delivered to the control room using a a Cat7 cable. In further embodiments, the force-sensing instrumentation includes a low noise circuit that is operable while in the presence of a magnetic field produced during the performing a magnetic resonance imaging scan.

Exemplary embodiments of the present disclosure also provide: a method of quantitative 31-phosphorus magnetic resonance spectroscopy measurement in human vastus lateralis muscle; a method of comparing differences between normal glucose tolerant and type 2 diabetes mellitus subjects in resting concentrations of adenosine triphosphate, inorganic phosphate, phosphocreatine, intramyocellular lipids and creatine, and their relationship to measures of glycemic control, insulin resistance and mitochondrial density and; and a method of comparing differences between normal glucose tolerant and type 2 diabetes mellitus subjects in parameters during and after an exercise regimen and their relationship to measures of glycemic control, insulin resistance and mitochondrial density.

The present disclosure was developed in an effort to address shortcomings in conventional MR-compatible ergometers by developing a repeatable MRS exercise protocol as a basis for performing ³¹P-MRS scans for research involving subjects with insulin resistance and type 2 diabetes mellitus (T2DM).

To achieve this goal, an exemplary embodiment of the present disclosure includes the design, development and prototyping of a safe, lightweight exercise apparatus, small enough to operate within the bore of the scanner and be compatible with the intense magnetic fields found inside a MRI system. The exercise apparatus provides resistance to the extension of the distal leg in order to exhaust the VL m. in a short period of time. To ensure the safety of the exercise, the use of massive weights is eliminated so that if the muscle gives out during the exercise period and the weight equivalent is dropped, both the subject and the scanner are protected from injury and/or damage.

In another exemplary embodiment, the apparatus was assembled and tested for functionality, comfort, safety and durability, and was able to accommodate a variety of subjects of different heights, weights, ages and physical abilities. Volunteers were fitted to the apparatus and a surface coil, configured as a unit, and performed the exercise protocol. Slice selective ³¹P-MRS data was collected on 11 subjects (5 female, 17-65 years old) with TR=3000 ms, NSA=2, BW=2200 Hz. The time for recovery of PCr values to their half-maximum (T-half) and/or the rate constant of recovery were used as indexes for the rate of ATP synthesis.

In another exemplary embodiment, the apparatus provides an indication of the relative amount of work being done by the subject during the exercise phase of the protocol. This was accomplished using strain gauge devices fused to the actuating bar where the force from the leg is applied. The work output data was captured by a digital acquisition module (DAQ) connected to the stain gauge circuitry and a Windows 7 computer. Software emulating a strip recorder was used to watch, in real-time, a subject's force of effort and the position of the actuating bar on the computer monitor. The software was configured to sample and collect data at a 50 Hz rate for 5 minutes and 20 seconds, which allows capturing a baseline 10 seconds before and after the 5 minutes exercise period. The data capture was written to an Excel compatible file for post processing.

In another exemplary embodiment, a method is provided to post process the exercise performance data to determine a relative quantity of work performed by each MRS study. Newtonian physics was applied to the data set to quantify a relative level of work imparted to the exercise apparatus.

In yet another embodiment, the present disclosure provides a surface coil holder having four degrees of freedom. The surface coil holder is capable of being attached to a scanner table of an exercise apparatus. The surface coil holder also includes a coil cradled in the coil holder and is secured by hook and loop strips. In further embodiments, the coil provides four degrees of freedom with adjustment in the orthogonal x, y, and z axes and one rotational adjustment on the z axis. In yet further embodiments; the surface coil holder is integrated into an exercise apparatus such that the coil is in a fixed position on an upper leg of a subject.

At least in one embodiment, the present disclosure provides an exercise apparatus for measuring work performed by a subject in a magnetic resonance imaging device, comprising: an actuating bar, an elastomer band positioned inside the actuating bar, a tower and roller bearing, and a pivot base supporting the actuating bar, the elastomer band positioned inside the actuating bar, and the tower and roller bearing. At least in one embodiment, the apparatus may further comprise at least four attach points to position, mount, and secure an apparatus to a secondary device. At least in one embodiment, the secondary device is a magnetic resonance imaging device. At least in one embodiment, the apparatus further comprises a magnetic resonance coil. At least in one embodiment, the magnetic resonance coil is positioned close to an isocenter of a magnet of the magnetic resonance imaging device, and the vastus lateralis muscle of the subject and a primary axis of the magnetic resonance coil are parallel to a magnetic field within a bore of the magnetic resonance imaging device. At least in one embodiment, a material is selected from a group consisting of polyvinylchloride (PVC), common PVC cement, and non-magnetic stainless steel, nylon, and combinations thereof. At least in one embodiment, the apparatus is adjustable to accommodate subjects from 60 inches to 77 inches in height. At least in one embodiment, the actuating bar has a single adjustment point to extend length of the actuating bar to match a subject's distal leg length. At least in one embodiment, the apparatus provides for the correct positioning of a leg of the subject. At least in one embodiment, the apparatus isolates movement of the leg in the y-axis direction. At least in one embodiment, the actuating bar comprises a pivot point with a slip bearing to withstand torque in two axes.

At least in one embodiment, the present disclosure provides a method for executing a magnetic resonance imaging protocol comprising: (a) positioning and attaching an apparatus to a scanner couch of a magnetic resonance imaging device; and (b) positioning a subject on the apparatus, wherein the apparatus comprises: a surface coil positioned close to an isocenter of a magnet of the magnetic resonance imaging device, an actuating bar, an elastomer band positioned inside the actuating bar, a tower and roller bearing, a pivot base supporting the actuating bar, the elastomer band positioned inside the actuating bar, and the tower and roller bearing, at least four attach points to position, mount, and secure the apparatus to the magnetic resonance imaging device; and a coil holder comprising the surface coil cradled in the coil holder and secured by a hook and loop strips.

At least in one embodiment, the present disclosure provides a force-sensing instrument for use in a magnetic resonance imaging device, comprising a strain gauge cemented to a surface of an actuating device that is operable in a magnetic resonance imaging device. At least in one embodiment, the strain gauge is a copper foil electrical circuit, which is bonded to a thin plastic film. At least in one embodiment, the strain gauge comprises two strain devices cemented to an actuating bar of the actuating device, and separated by about 180±2 degrees. At least in one embodiment, the two strain devices form a Wheatstone bridge circuit along with two precision resistors, such that the two strain devices are positioned to sense strain in only one axis from a torque in a bar thereof. At least in one embodiment, the force-sensing instrument further comprises a position indicator device. At least in one embodiment, the force-sensing instrument fits within an actuating bar of the actuating device. At least in one embodiment, the present disclosure provides a magnetic resonance imaging device comprising a force-sensing instrument, wherein the actuating device is adjustable to accommodate subjects from 60 inches to 77 inches in height.

At least in one embodiment, the present disclosure provides a method of using a force-sensing instrumentation, comprising the steps of: attaching the force-sensing instrumentation to an ergometer attached to the scanner table of a magnetic resonance imaging device; adjusting the force-sensing instrumentation to accommodate a subject; operating the force-sensing instrumentation by the subject; delivering data obtained by the force-sensing instrumentation to a control room; performing a magnetic resonance imaging scan of the subject; delivering data obtained from the magnetic resonance imaging scan of the subject to the control room; further wherein the step of operating the force-sensing instrumentation and the step of performing a magnetic resonance imaging scan overlap. At least in one embodiment, the method further comprises the steps of: determining a relative amount of work being done by the subject while operating the force sensing instrumentation based on the data obtained by the force-sensing instrumentation and the data obtained by the magnetic resonance imaging scan. At least in one embodiment, data obtained by the force-sensing instrumentation is delivered to the control room using Cath RJ45 connector jacks. At least in one embodiment, data obtained by the force-sensing instrumentation is delivered to the control room using a Cat7 cable. At least in one embodiment, the force-sensing instrumentation comprises: a low noise circuit that is operable while in the presence of time varying magnetic fields produced during the performing a magnetic resonance imaging scan.

At least in one embodiment, the present disclosure provides a surface coil holder having four degrees of freedom, capable of being attached to a scanner table of an exercise apparatus, wherein the surface coil holder comprises a coil cradled in the coil holder and secured by hook and loop strips. At least in one embodiment, the coil provides four degrees of freedom with adjustment in the orthogonal x, y, and z axes and one rotational adjustment on the z axis. At least in one embodiment, the surface coil holder is integrated into an exercise apparatus such that the coil is in a fixed position on an upper leg of a subject. At least in one embodiment, the surface oil holder is constructed of materials which are compatible with an MRI scanner of at least 3 Tesla. At least in one embodiment, the surface coil holder is constructed such that it does not interfere with the homogeneity of a B₀ field or functional operation of a scanner. At least in one embodiment, the surface coil holder holds a surface coil that is a dual tuned ¹H-³¹P TX/RX surface coil. At least in one embodiment, the surface coil holder is adjustable to fit surface coils of different outside dimensional curvatures.

### BRIEF DESCRIPTION OF DRAWINGS

These and other features, aspects, and advantages of the disclosure are better understood with regard to the following Detailed Description, appended Claims, and accompanying Figures. It is to be noted, however, that the Figures illustrate only various embodiments of the disclosure and are therefore not to be considered limiting of the invention's scope as it may include other effective embodiments as well.

FIG. 1 is a schematic and ball model representation of the chemical structure of phosphocreatine (C₄H₁₀N₃O₅).

FIG. 2 is a schematic and ball model representation of the chemical structure of creatine (C₄H₉N₃O₂).

FIG. 3 is an example of a ³¹P-MRS spectrum displayed on the Siemens Magnetic Resonance scanner.

FIG. 4 is a photograph of an exemplary embodiment of the apparatus of the present disclosure wherein the elastomer is exposed.

FIG. 5 is a photograph of an exemplary embodiment of the apparatus of the present disclosure as the apparatus enters the scanning bore of the Siemens Magnetic Resonance scanner.

FIG. 6 is a photograph of a subject positioned in a prone, head first position on a scanner couch, entering the Siemens Magnetic Resonance Scanner.

FIG. 7 is a photograph of an exemplary embodiment of the coil holder of the present disclosure without the ¹H-³¹P dual tuned surface coil positioned thereon.

FIG. 8 is a photograph of an exemplary embodiment of the coil holder of the present disclosure with the ¹H-³¹P dual tuned surface coil positioned thereon.

FIG. 9 is a photograph of an exemplary embodiment of the apparatus of the present disclosure showing the attachment points at which the apparatus is positioned and locked to the scanner table of the Siemens Magnetic Resonance Scanner.

FIG. 10 is a photograph of an exemplary embodiment of the apparatus of the present disclosure showing the electrical connections thereof.

FIG. 11 is a photograph of an exemplary embodiment of the apparatus of the present disclosure showing the correct position of the subject and the surface coil on the scanner couch.

FIG. 12 is a photograph of an exemplary embodiment of the apparatus of the present disclosure showing the lowest position of the apparatus during the five minute exercise period. Here the elastomer is exposed and is extended to its maximum position.

FIG. 13 is a photograph of an exemplary embodiment of one strain gauge of the present disclosure positioned on the actuating bar, wherein the elastomer is exposed.

FIGS. 14A and 14B are photographs of an exemplary embodiment of the apparatus of the present disclosure showing the instrumentation amplifier assembly. In FIG. 14A, the PCB is mounted with SMD components, and in FIGS. 14A and B, the circuit contains a low pass filter, two legs of the Wheatstone bridge, and power conditioning and connections to the shielded RJ45 jack. The circuit board is inserted into the PVC tubing and is capped with the connector shown in FIG. 14B. Fine wires attach from the circuit to the two strain gauges.

FIG. 15 is a schematic visualization of the working concept behind an exemplary embodiment of the present disclosure, which includes a strain gauge on a material under exaggerated bending.

FIG. 16 is a schematic representation of a Wheatstone bridge circuit.

FIG. 17 illustrates the physical features of a resistive foil type strain gauge according to an exemplary embodiment of the present disclosure and a typical strain gauge (bottom photograph). The dimensions (in inches) of a typical strain gauge include gauge length=0.125, gauge width=0.100, overall length=0.275, and overall width=0.120.

FIG. 18 is a schematic representation of a strain gauge system.

FIG. 19 is a graphical representation of the exercise apparatus work data of an exemplary embodiment of the apparatus of the present disclosure.

FIG. 20 is a graph showing the strain gauge output as a function of the weight applied (weight equivalent) to the articulating bar.

FIG. 21 is a graph showing the resistance tension (weight equivalent) as a function of position over a range of motion. This graph shows the calibration data of resistive tension (weight equivalent) over the range of motion of the articulating bar.

FIG. 22 is photograph showing an exemplary embodiment of the apparatus of the present disclosure, and a subject positioned therein, wherein the elastomer is exposed. This photograph shows the correct position of the subject inside the bore of the Siemens Trio scanner, with the foot lifted to the top of the bore allowing for about 40 degrees of movement range available for leg extensions.

FIG. 23 is a schematic representation of the phosphocreatine circuit model of intracellular energy. The phosphocreatine (PCr) circuit shows the re-phosphorylation of creatine (Cr) by using mitochondrial creatine kinase (MtCK) in mitochondria using ATP derived from oxidative phosphorylation (Oxid phos) and subsequent use of mitochondrial PCr by cytosolic creatine kinase (CK) to resupply ATP for muscle activity. (Source: OpenI.)

FIG. 24 is an example of PCr levels observed during the MRS exercise protocol.

FIG. 25 is a mechanical drawing of a side view an exemplary embodiment of the apparatus of the present disclosure (dimensions in inches).

FIG. 26 is a mechanical drawing of a front view of an exemplary embodiment of the apparatus of the present disclosure (dimensions in inches).

FIG. 27 is a mechanical drawing of a top view of an exemplary embodiment of the apparatus of the present disclosure (dimensions in inches).

FIG. 28 is a representation of an external reference vial of MDP and the H₃PO₄ leg phantom. In FIG. 28, (A) is the external reference vial of MDP, (B) is the MRI leg surface coil in the custom holder, and (C) is the H₃PO₄ leg phantom shown on the custom coil holder. The ¹H/³¹P dual-tuned spectroscopy coil is fixed to the custom coil holder which was adjustable in three dimensions. The 6 mL plastic vial of MDP in (A) is attached to the center of the coil with padding surrounding it to prevent compression or movement. After a subject is scanned, the 4 L, 35 mM H₃PO₄ phantom simulating a human leg (B) and (C) is placed on the coil.

FIG. 29 is a representation of the scan protocol for an absolute quantitation protocol. In this protocol: (A), first the ³¹P-MRS slab is positioned in the subject over the vastus lateralis avoiding excess subcutaneous fat; (B) the slab is placed at the same position in the leg phantom to maintain the same region in the B1 field of the RF coil; (C) another spectrum is taken of the MDP external reference vial with the subject placed in the coil; and (D) a spectrum of the external reference vial is acquired while the phantom is in the coil. The ratio of these MDP scans corrects for differences in coil loading.

FIG. 30 is a representation of the volume correction for the ³¹P-MRS protocol. In FIG. 30: (A) a five-slice MRI sequence is obtained with the same center location as the spectroscopy slab shown in FIG. 29. The area of the muscle tissue within each slice is found and a simple geometrical equation is used to calculate the approximate volume of muscle tissue within the slab; and (B) depicts the top slice from the localizer image (A).

FIG. 31 demonstrates ³¹P-MRS chemical shift imaging, where a ³¹P-MRS Chemical Shift Imaging (CSI) is used to evaluate tissue sources in vivo. Here, a proton magnetic resonance image of a human leg is overlaid with a ³¹P-MRS chemical shift image. Percent of total PCr signal is indicated in each voxel relative to the maximum PCr signal. No PCr signal was recorded from the bone and only a small amount (3%) of the total signal came from the voxels containing fat. The small contribution from fat is attributed to a partial volume effect from the voxels also containing a small amount of muscle tissue.

FIG. 32 are scatter plots depicting relationships of baseline [PCr] to [ATP], HbA1c and FPG for all subjects. There are significant correlations between the [PCr] and (A) [ATP] (r=0.82, p<0.001), (B) HbA1c (ρ=−0.51, p=0.01) and (C) fasting plasma glucose (FPG) levels (ρ=−0.63, p<0.001).

FIG. 33 are baseline PCr plots versus IMCL concentrations. The correlation between [PCr] and [IMCL] reverses based on disease status. (A) T2DM: (r=0.72, p=0.04) and (B) NGT: (r=−0.77, p=0.02).

FIG. 34 shows correlations of mitochondrial density with resting Phosphate metabolites. The groups are shown with the red circles indicating the NGT subjects and the blue diamonds indicating the T2DM subjects. Significant correlations were found in all subjects as well as both groups for all three metabolites: (A) [PCr] (r=−0.67, p=0.009), (B) [Pi] (r=−0.61, p=0.02), and (C) [ATP] (r=−0.63, p=0.02).

FIG. 35 provides representative electron microscopy images of a NGT and a T2DM Subject. The electron microscopy images show a loss of muscle structure and regularity in T2DM subjects (B). In NGT subjects (A) the mitochondria are structured in neat pairs along the z line.

FIG. 36 shows a front view of a coil holder in accordance with an embodiment of the present disclosure.

FIG. 37 shows a side view of a coil holder in accordance with an embodiment of the present disclosure.

FIG. 38 shows a top view of a coil holder in accordance with an embodiment of the present disclosure.

### DETAILED DESCRIPTION

Advantages and features of the present disclosure and methods of accomplishing the same will be apparent by referring to embodiments described below in detail in connection with the accompanying drawings. However, the present disclosure is not limited to the embodiments disclosed below and may be implemented in various different forms. The embodiments are provided only for completing the disclosure of the present disclosure and for fully representing the scope of the present disclosure to those skilled in the art.

For simplicity and clarity of illustration, the drawing figures illustrate the general manner of construction, and descriptions and details of well-known features and techniques may be omitted to avoid unnecessarily obscuring the discussion of the described embodiments of the disclosure. Additionally, elements in the drawing figures are not necessarily drawn to scale. For example, the dimensions of some of the elements in the figures may be exaggerated relative to other elements to help improve understanding of embodiments of the present disclosure. Like reference numerals refer to like elements throughout the specification.

Hereinafter, embodiments of the present disclosure will be described with reference to the accompanying drawings. In assigning reference numerals to elements in the drawings, the same elements will be designated by the same reference numerals as far as possible although they are illustrated in different drawings. Further, in the following description of the present disclosure, a detailed description of known functions and configurations incorporated herein will be omitted when it may make the subject matter of the present disclosure rather unclear.

In addition, terms, such as first, second, A, B, (a), (b) or the like may be used herein in describing elements of the present disclosure. Each of these terminologies is not used to define an essence, order or sequence of a corresponding component but used merely to distinguish the corresponding component from other component(s). In the case that it is described that a certain element “is connected to”, “is coupled to”, or “is connected with” another element, it should be understood that not only can the certain element be directly connected or coupled to the another element, but an additional element may also be “interposed” between the elements or the elements may be connected or coupled to each other through an additional element.

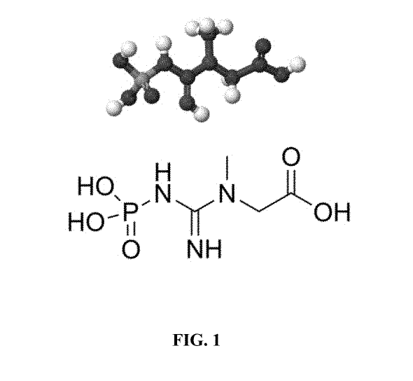

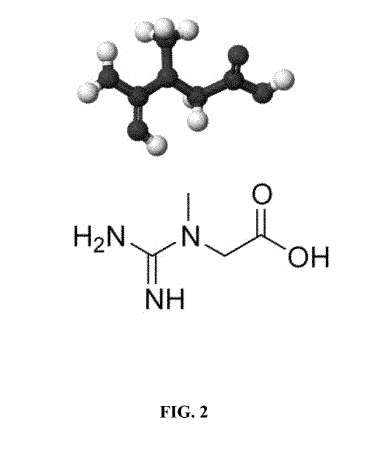

The measurement of adenosine triphosphate (ATP) synthesis via mitochondrial function is key to the proper function of skeletal muscle, which is an important topic in aging and diabetes studies and is also useful for sports medicine and athlete training. During the contraction of skeletal muscles, ATP is converted to adenosine diphosphate (ADP) by hydrolysis and this consumption of energy is accompanied by a rise in inorganic phosphate (Pi). Phosphocreatine (PCr) (C₄H₁₀N₃O₅P) is a fast source of energy that can be used immediately for ATP synthesis via the forward creatine kinase (CK) reaction in tissue such as skeletal muscle, which may have high and fluctuating energy demands. FIG. 1 provides a chemical structure of PCr and FIG. 2 provides a chemical structure of creatine.

Sustained exercise of large muscles such as the vastus lateralis results in a substantial drop in the level of PCr as ATP is immediately produced and consumed again during exercise. As the level of PCr in the muscle drops, the speed of ATP resynthesis slows and the concentration of Pi rises. Muscle output is reduced and muscle contractions are not as strong. During a period of non-exercise (recovery), PCr is replenished by the re-phosphorylation of creatine as ATP is produced in the mitochondria. The organic chemistry equations for ATP to ADP and PCr to Pi as follows:

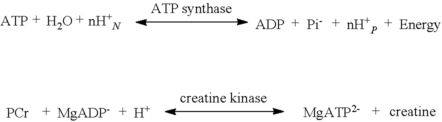

In the above equations, the first illustrates the bidirectional process of the hydrolysis of ATP to ADP and inorganic phosphate, and the second illustrates the bidirectional process of the breakdown and re-synthesis of PCr. Both reactions are reversible but the reverse ATP synthase reaction does not typically occur under normal physiological conditions.

Since the process of PCr recovery is a well-characterized in vivo biomarker, its measurement can provide insights into the effects of metabolic diseases and the effects of aging. The use of ³¹P magnetic resonance spectroscopy (³¹P-MRS) provides a non-invasive tool to measure the rate of PCr recovery which is presumed to be proportional to the rate of ATP synthesis after moderate exercise.

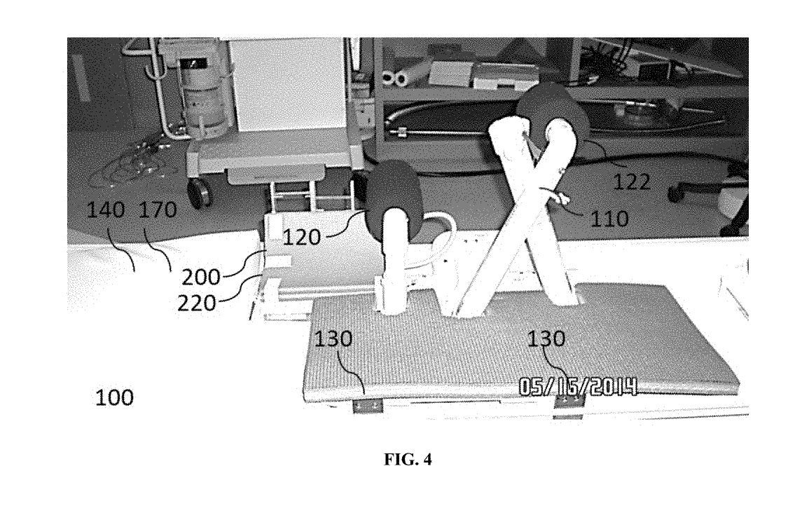

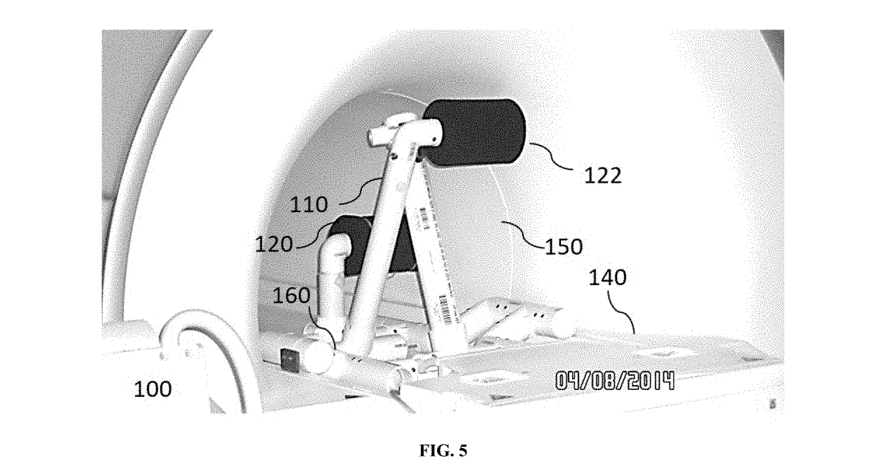

Referring to FIGS. 4 and 5, in an exemplary embodiment of the present disclosure, a reproducible MRS exercise system and a protocol to acquire reproducible ³¹P-MRS measurements of PCr recovery that would be usable and reproducible in a wide range of subjects were developed. Such an apparatus would be compatible, for instance, with a 3T Siemens Trio MR scanner and a dual-tuned ¹H-³¹P TX/RX surface coil fixed over the VL m. In addition, a real time data collection system to record the amount of work or energy a subject generated during exercise was integrated into the exercise device.

In another exemplary embodiment of the present disclosure, a prototype of an exercise apparatus, the surface coil and exercise technique were designed and developed, and integrated with the MR scanner. In order to correlate PCr measurements with the muscular output, the apparatus was instrumented with a strain gauge circuit and an instrumentation amplifier to measure force in one axis. Using a small software program to emulate a strip recorder, the strain gauge was sampled every 20 milliseconds by an analog data acquisition (DAQ) device, converted to digital (ADC) and stored as an Excel file. In addition to the force measurement, a second channel recorded the position of the leg relative to the full range of motion of the apparatus's actuating bar.

Nuclear Magnetic Resonance (NMR) is a form of spectroscopy, which can detect the magnetic moment of nuclei by placing chemical compounds in a strong external magnetic field. Nuclei in the magnetic field will absorb and emit electromagnetic radiation at a unique resonance frequency according to the type of nucleus and to its molecular composition. Each chemical compound produces a chemical frequency shift in the NMR spectra by a small induced energy difference due to the magnetic shielding of electrons in the molecule. The low energy absorption of MRS makes it a perfect technique for in vivo spectral analysis since it is nondestructive and noninvasive. The first reported in vivo use of ³¹P-MR spectroscopy was in 1974. Certain elements are better for MRS due to the amplitude of the signal they produce. Suitable nuclei include ¹H, ¹³C, ¹⁵N, ²³Na and ³¹P. For these nuclei, a spin magnetic moment is present which causes the nuclei to precess with its rotational axis aligned with the large static magnetic field (B₀).

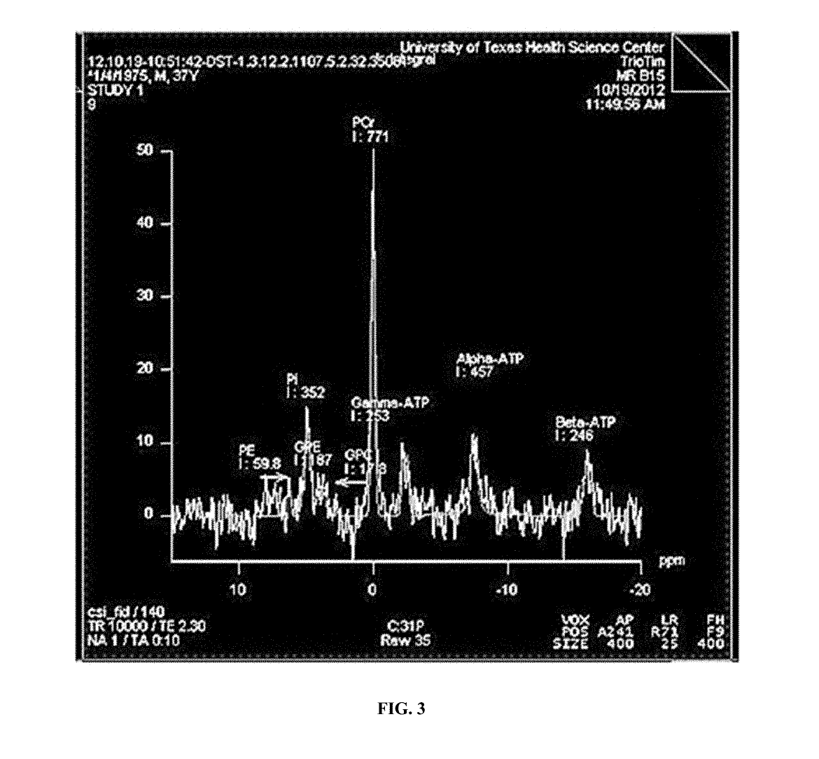

For ³¹P-MRS a resonant excitation center frequency of 49.89 MHz is used to produce the spectra. In an exemplary embodiment, the amplitudes of the chemical shifts of PCr and Pi were of interest. Chemical shifts are referenced to 85% concentration of phosphoric acid via a solution of methylenediphosphonic acid (MDP) (CH₆O₆P₂), which is assigned the chemical shift of zero. The chemical shift of PCr is 3.93 ppm and the shift of Pi is 8.95 ppm or 5.03 ppm when the PCr peak is centered at 0 ppm. FIG. 3 shows a sample spectrum of these shifts wherein the highest peak corresponds to PCr.

Due to the phosphorous nuclei being studied and the relatively low signal to noise ratio (SNR), a specially designed coil is used for ³¹P-MRS. The coil used in the present disclosure is a dual-tuned ¹H-³¹P TX/RX surface coil. This coil is placed in very close proximity to the VL m. because the sensitive volume of the coil is small and the spectral signal is small. In some embodiments, a coil manufactured by Rapid Biomedical GmbH of Rimpar, Germany is used.

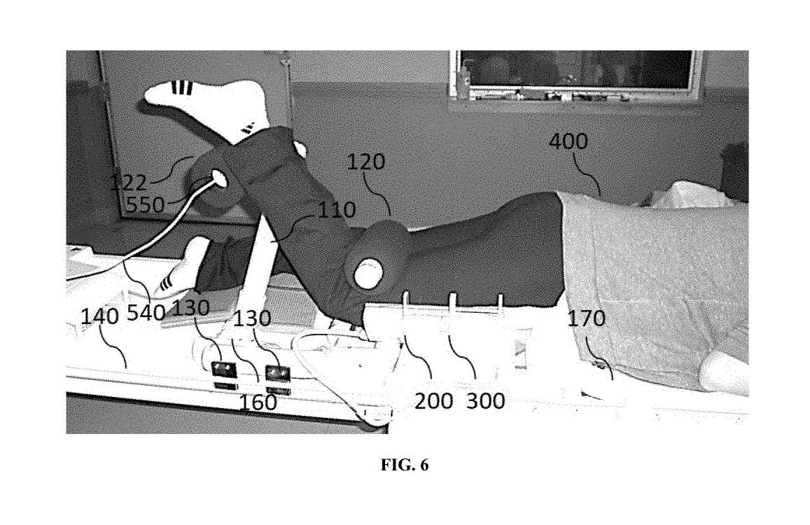

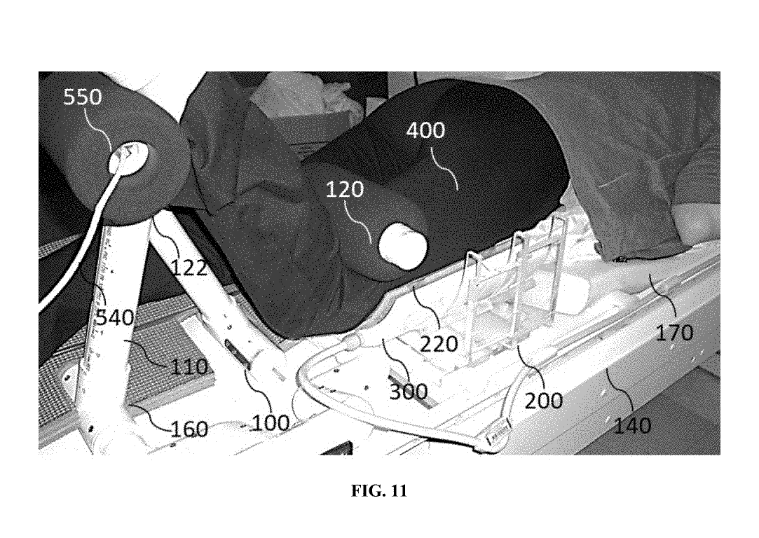

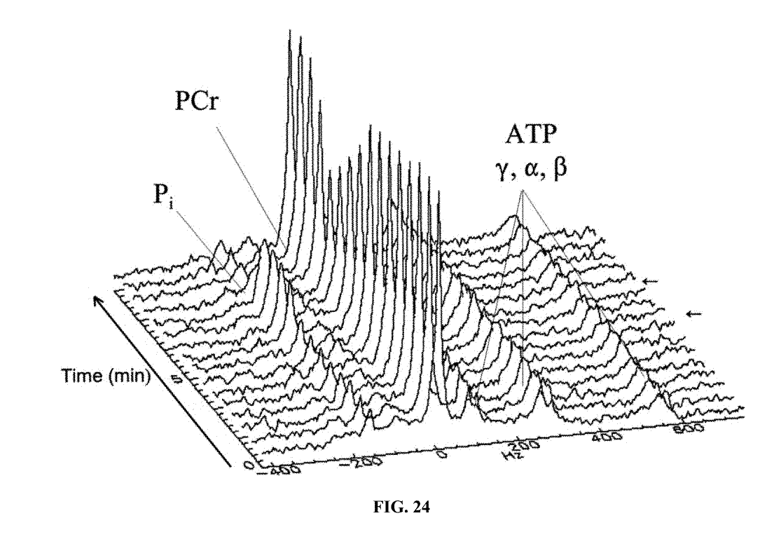

In an exemplary embodiment of the present disclosure, MRS tests were run to determine the optimal flip angle and time of repetition (TR). As a result of those tests, it was decided to design an exercise apparatus to accommodate subjects in a supine, feet first position since the SNR appeared higher. Referring to FIGS. 6 and 11, it was determined later, a subject in a prone, head first position produced a better SNR and superior spectral resolution. This was attributed to the location and angular position of the surface coil with respect to the B₀ field. The prone subject placed the coil closer to the isocenter of the magnet, and the VL m. and the axis of the saddle coil were both closely parallel to B₀. A sample ³¹P-MRS spectrum showing spectra of PCr referenced to zero and Pᵢ with a 5.03 ppm chemical shift is shown in FIG. 24. This baseline measurement taken before the start of the exercise period was acquired with one acquisition and a 3 second inter-acquisition interval. Visible spectral peaks, from left to right, include phosphodiesters (PE) at ˜6 ppm, inorganic phosphate (Pi) at ˜5 ppm, glycerolphosphoethanolamine and glycerolphosphocholine (GPE and GPC) at ˜3 ppm and ˜4 ppm, phosphocreatine (PCr) at 0 ppm, and the ATP resonances, γATP (−2.5 ppm), αATP (−8 ppm) and βATP (−17 ppm).

In an exemplary embodiment, both supine and prone prototypes were built and tested. Both designs achieved the end goal of exercising the VL m. to a point of near PCr depletion. The prone device was the preferred configuration and allowed favorable positioning of the coil and produced a higher SNR.

In another exemplary embodiment, a method to quantify the VL m. work output was devised to correlate the drop in PCr to the amount of work done by a subject. The apparatus was instrumented with a strain gauge with appropriate support circuitry and a computer interface. A small software program which emulated a strip recorder was used to monitor the amount of force the subject used to extend the lower leg at the knee (extension) and the duration of the force applied by the subject.

In another exemplary embodiment, a second circuit was added to monitor and record the instantaneous position of the actuating bar, indicating where the subject's leg was in relation to the full range of motion available. With the position data, one could determine whether subjects were able to fully extend their distal leg as fatigue set in and to what extent the subject found it difficult to push the bar fully down to the stop position. Variations in exercise techniques and patterns became apparent from subject to subject.

A ¹H scout image was first made of the VL m. with the dual tuned surface coil. Using the scout, a volume slab was defined within the VL m. for the ³¹P-MRS acquisitions. The slice-selective ³¹P-MRS signal acquisition was performed on eleven subjects (age 17-65, 5 female) with a TR=3000 ms, NSA=2, BW=2200 Hz, and a 90 degrees flip angle. The MRS exercise protocol consisted of a two minute baseline period where the subject remained still, followed by a five minute period of sustained, periodic exercise, which was followed by an eight minute recovery period. A total of 150 spectra were obtained per study.

The protocol ran with 3 seconds (2 averages) between sampled spectra. The subject was asked to coordinate their leg extensions so they were performed and released between samples with a 3 second repetition rate. Synchronization was accomplished by the subject listening for the Lorentz noise from the gradient coil pulses as a trigger for performing the leg extension, and returning the leg to the relaxed position within one second. Although this coordination was desirable, it was not critical to the data collected. Consistence of the PCr and Pi data was improved by acquiring spectra while the leg was most stationary relative to the surface coil.

The spectral data were removed from the scanner as raw data exports, one sample at a time and transferred to a PC where Java magnetic resonance user interface (jMRUI) and the R statistical software package were used for post processing and data analysis. By fitting the constant D in the equation below, the recovery rate constant, k, was calculated as a rate of PCr and Pi levels recovering to their baseline values. This data is used as an index for the rate of ATP synthesis. PCr recovery data were fit to the function:

PCr(t)=PCr(0)+D*[1−e^{(-kt)}]

by using the Levenberg-Marquardt algorithm (nlsLM function) in the R statistical package, where D and k were the fitted variables. The nlsLM function is used for nonlinear regression variable fitting using the Levenberg-Marquardt (L-M) algorithm vs. the Gauss-Newton algorithm with a partial linear fitter. The summation of residuals is sum of squares minimized using the LM algorithm.

A discussion of the features and assembly of an exemplary embodiment of the present disclosure follows. In general, the apparatus is compatible with clinical MRI scanners. No materials may be used which are magnetic in nature or capable of producing large inhomogeneities in the magnetic fields. Although non-magnetic, certain materials are avoided since susceptibility artifacts could be produced in the magnetic and electromagnetic (RF) fields generated by the scanner and surface coil. The apparatus is unaffected by the scanner and in turn would cause minimal inhomogeneities in the B₀ field.

The materials chosen for the prototype rigid structures were polyvinylchloride (PVC), common PVC cement, and non-magnetic stainless steel and nylon fasteners. To provide the resistive force, a common elastomer band was chosen of appropriate length. A performance grade acrylic plastic was used for the attach points to mount the apparatus to the scanner's couch. For comfort, commercial grade foam rollers were used at locations that the subject interfaced to the apparatus. The use of aluminum, brass, and ferrous materials was avoided.

Various exemplary embodiments of the present disclosure are described as follows.

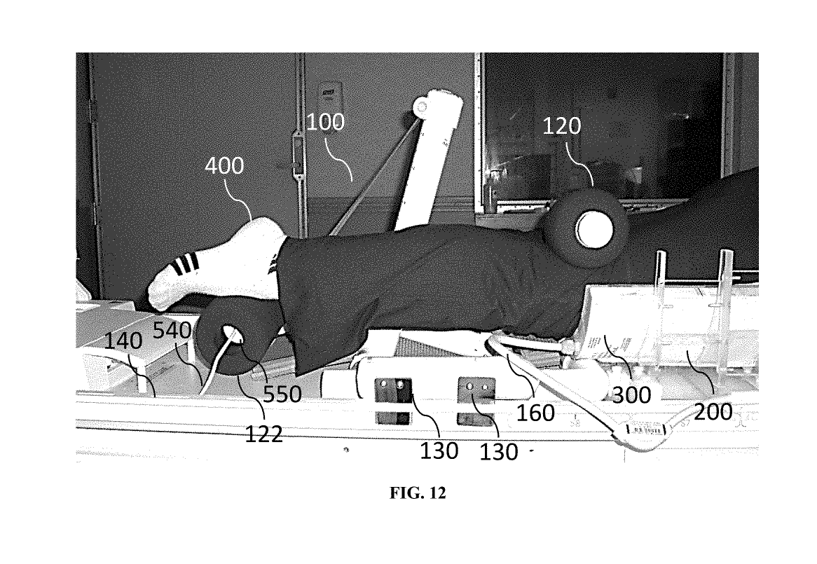

In an exemplary embodiment, the apparatus 100 provides an appropriate amount of exercise resistance (i.e. weight) to fatigue the VL m. within a five minute exercise window for most subjects, such as repeating sequences shown in FIGS. 11 and 12. The elastomer chosen provides a near linear elastic force according to Hooke's law (k varies from 110 to 160 N/m). The band was internally routed through the device so its length provided between 0 to 11 equivalent kilograms-force of resistance (100 N) (24.25 lbs.-force).

In another exemplary embodiment, the apparatus 100 uses elastomer bands to provide resistance and eliminates the need for massive weights. Elastomer bands have numerous advantages. Using lead or sand means the force of resistance would be provided by the acceleration of gravity. To supply enough resistance, a large amount of heavy material would be needed. With the limited space within the bore 150 of the scanner, a tension cable would have been required since the location of the weights would be limited. Elastomers provide tension due to Hooke's law and does not rely on gravity, so the position and the resistive vector is not constrained.

In another embodiment, the apparatus 100 provides for the safety of the subject 400 and the scanner. During the exercise phase the goal is to quickly deplete the PCr and fatigue the muscle. If a subject 400 suddenly lost control of the load only a small mass would be dropped. This concept is demonstrated in health clubs where many patrons avoid heavy free weights. Injuries have occurred when one loses control while lifting a heavy mass. It is also possible that the scanner could potentially be damaged by such an event.

In another embodiment, the apparatus 100 is adjustable to accommodate subjects 400 from 60 inches to 77 inches in height. The actuating bar 110 has a single adjustment to extend the length of the bar to match the subject's distal leg length.

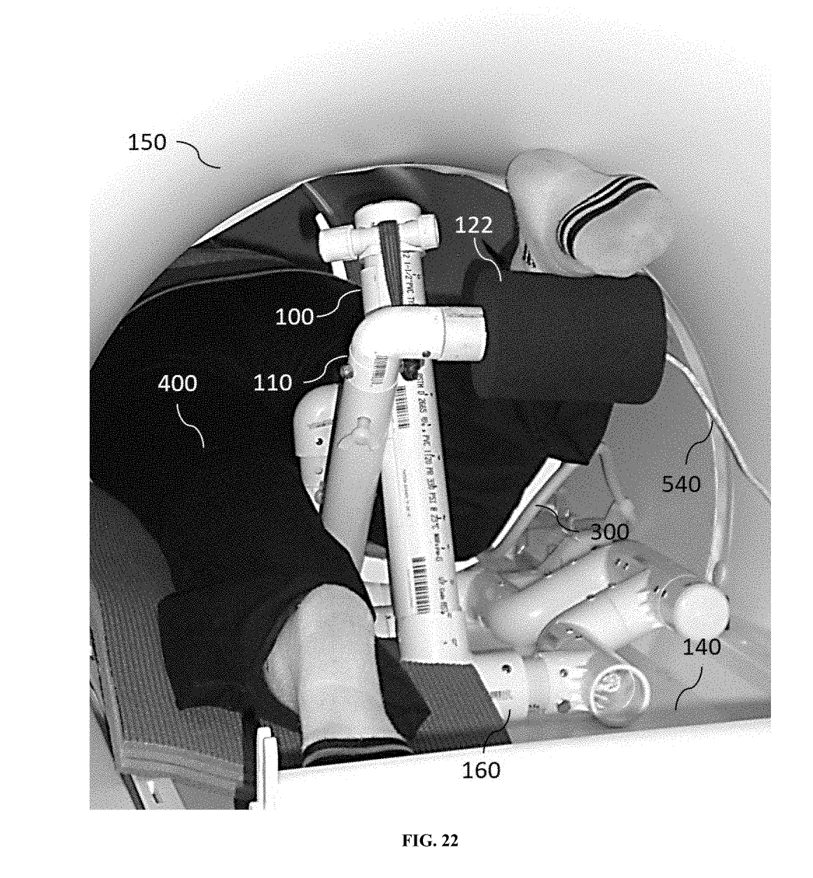

Referring to FIGS. 6, 11, 12 and 22, in another embodiment of the present disclosure, the apparatus 100 provides for the correct positioning of the right leg. By design, the device positions the subject's leg so that the VL m. is near magnet isocenter and extension of the VL actuates the mechanism, as shown in FIG. 22. The design isolates leg movement in the y-axis direction. In the prone position, the resistive load and flexion/extension movement causes the undesirable effect of lifting the thigh and pelvis slightly off the couch 170. To provide a solid leverage point, a pad 120 is locked over the posterior leg just superior to the popliteal fossa. This pad 120 provides a secondary benefit of holding the thigh tightly to the surface coil 300.

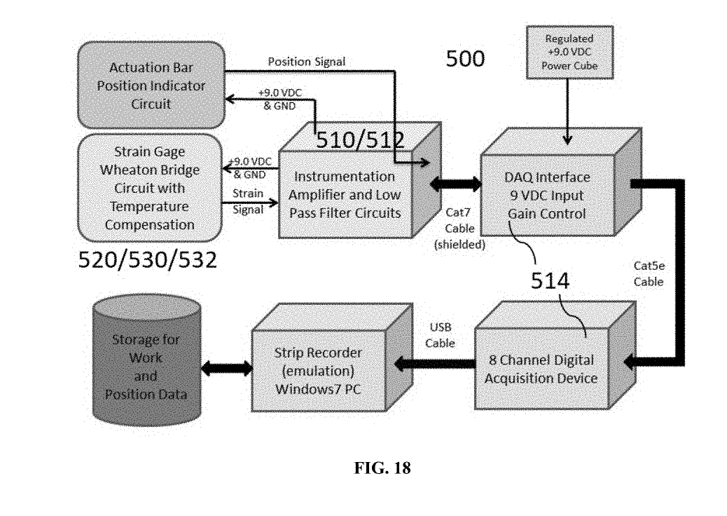

Referring to FIG. 18, in another embodiment of the present disclosure, the apparatus 100 is equipped with instrumentation 500 to provide performance data of the subject's exercise period and the amount of work performed. A strain gauge circuit 530 and instrumentation amplifier 510 were designed and built into the actuating bar 110 of the apparatus 100. A data acquisition device (DAQ) with analog to digital conversion (ADC) capabilities interfaced the instrumentation amplifier 510 via a Cat7 twisted pair Ethernet cable 540. A computer running an emulated strip recorder program captured data every 20 milliseconds.

In another exemplary embodiment of the present disclosure the apparatus 100 provides for the subject's comfort. Due to the extra time needed to create a scout image, suppress the water signal, shim the B₀ field and position the desired slab (region of interest, ROI) for MRS, the subject 400 is in position for one hour. Issues with transient paresthesia (pins and needles) of the foot and the distal leg required identifying and eliminated pressure points using pads found on commercial exercise equipment.

In another exemplary embodiment of the present disclosure, the apparatus 100 provides a smooth operating experience. Low friction materials and lubricants were used at actuating points. PVC parts create a little frictional heat when rubbed together. The slight heating is enough to soften the material making it slightly tacky. An appropriate lubricant is identified for each friction point so the actuating point moves freely.

In another exemplary embodiment of the present disclosure, the apparatus 100 is compact and lightweight. With the elimination of massive parts the total weight of the apparatus 100 is 4.68 kg. The length, width and height of the apparatus 100 are 48.9 cm, 53.0 cm, and 42.8 cm respectively.

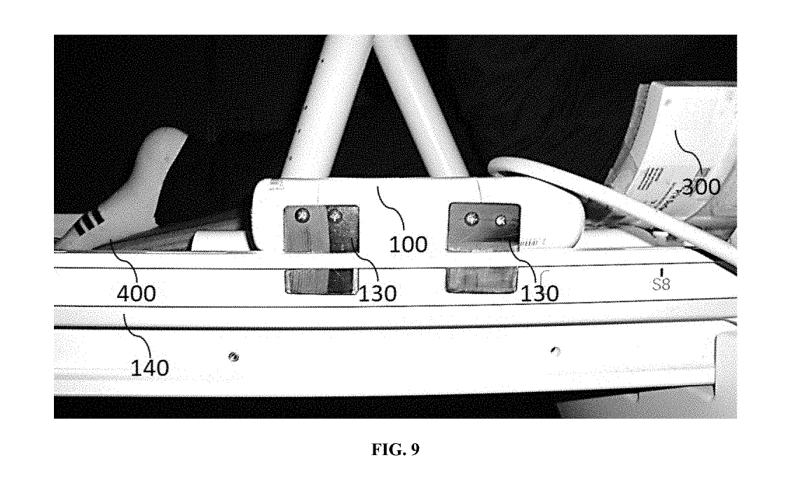

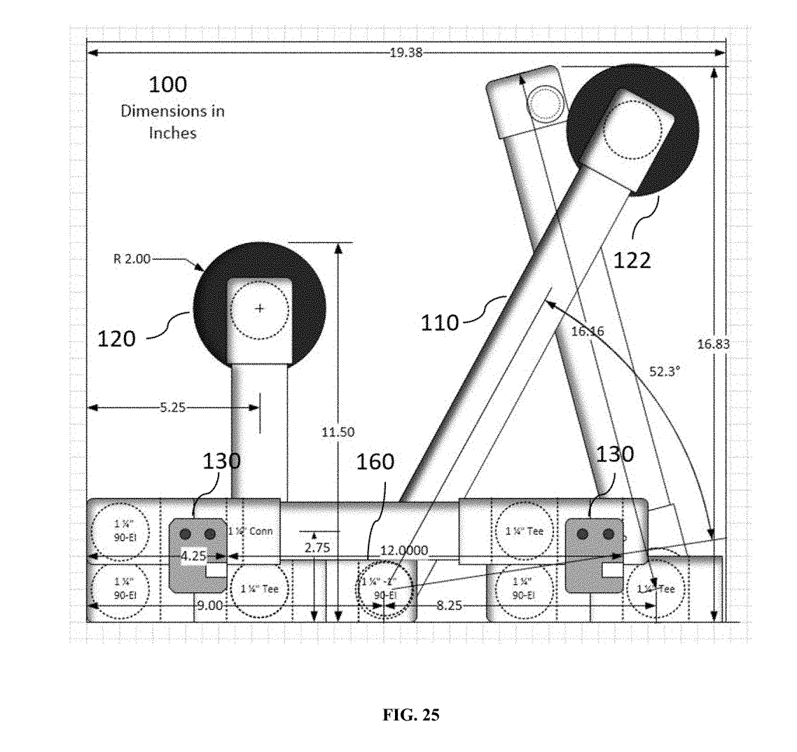

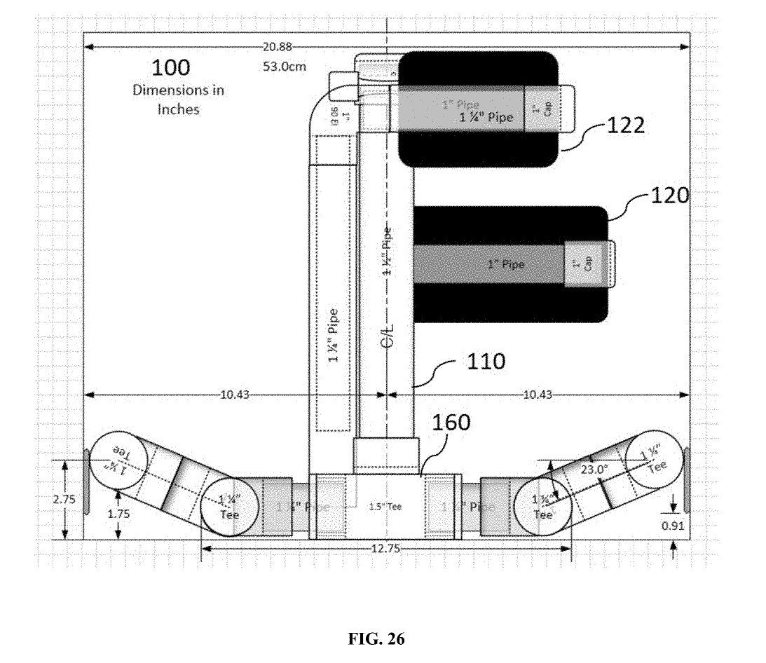

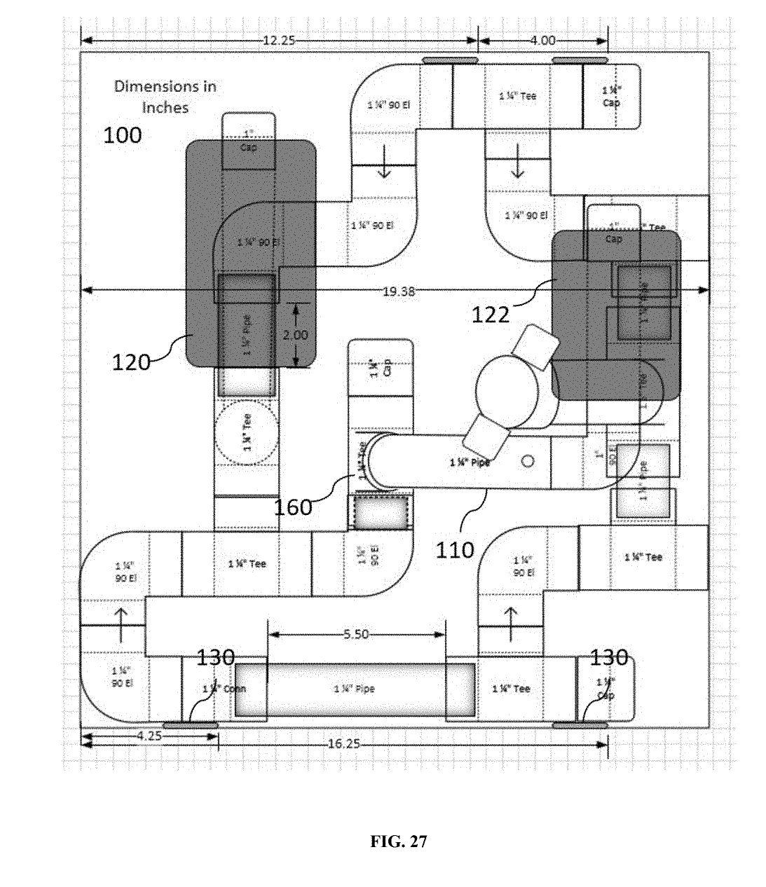

In another exemplary embodiment of the present disclosure, the apparatus allows for quick and easy setup. Referring to FIG. 25, the apparatus has four attach points 130 to position, mount, and secure the device to the scanner. Notched acrylic parts were machined to position the apparatus correctly. The apparatus is locked into place by pushing the apparatus in the direction of the z axis, as shown in FIG. 9. Still referring to FIG. 9, the four attach points 130 are used to position, mount, and secure the device to the scanner.

In another exemplary embodiment of the present disclosure, the apparatus is installed by a single individual. Setup time is less than five minutes including mounting the surface coil 300.

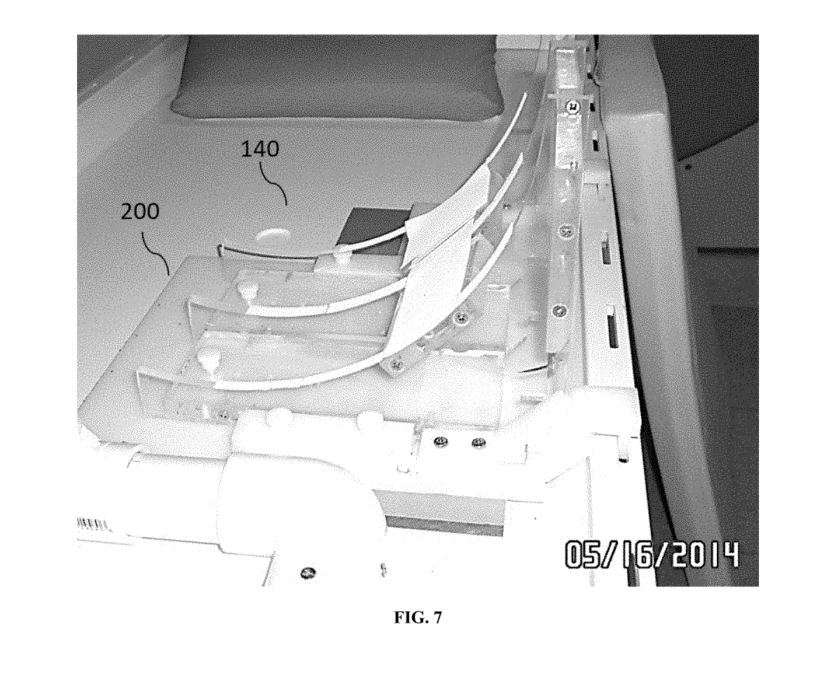

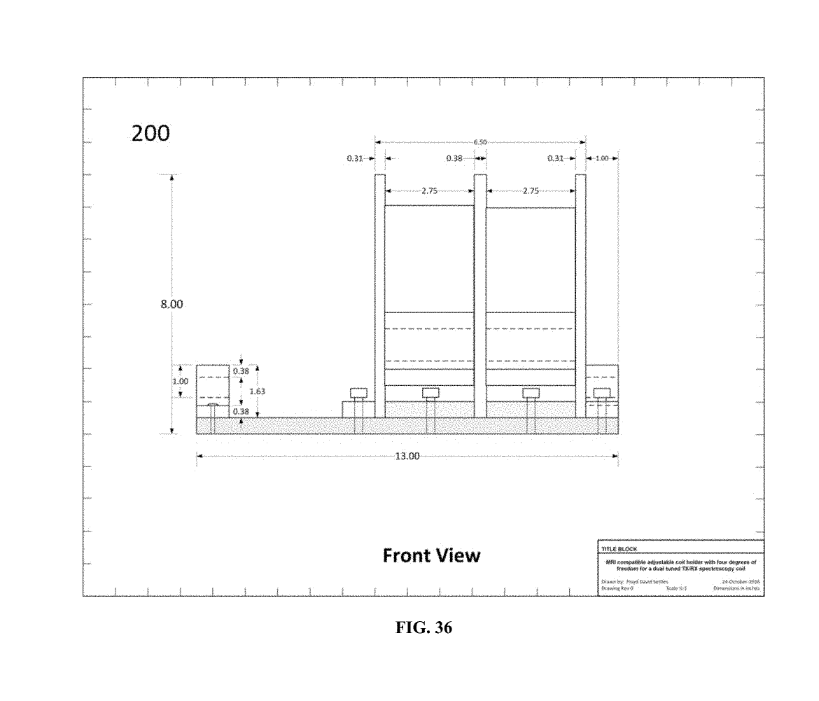

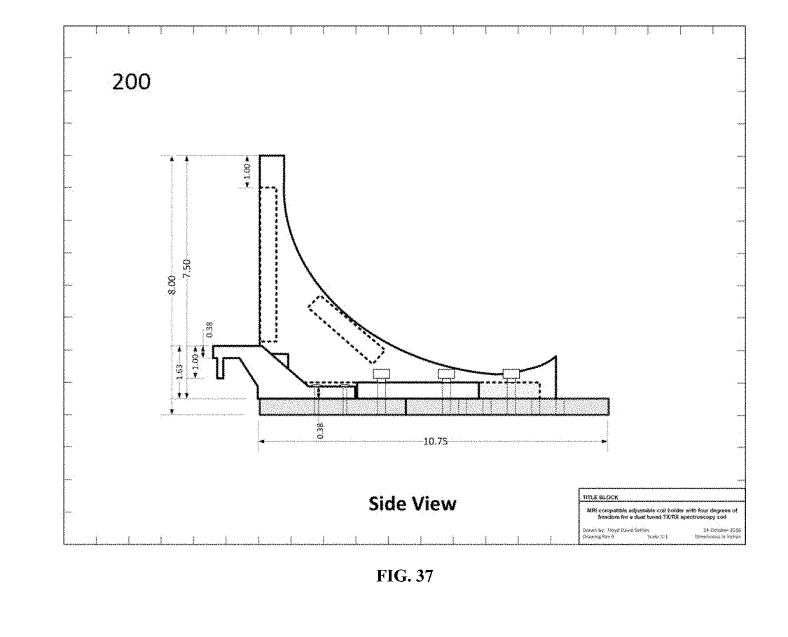

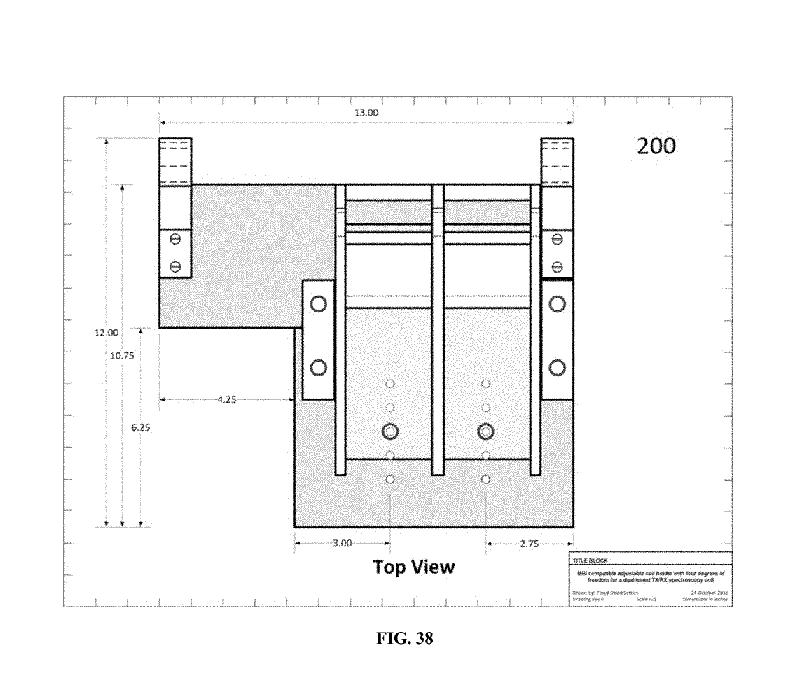

In another exemplary embodiment of the present disclosure, the apparatus 100 provides for the reliable and repeatable positioning of the surface coil 300. A custom built surface coilholder 200 was designed, machined and assembled in-house. The holder 200 attaches to the scanner table 140 just prior to the exercise apparatus 100. The coil 300 is cradled in the holder 200 and secured by hook and loop strips. The coil holder 200 provides 4 degrees of freedom with adjustments in the orthogonal x, y, and z axes and one rotational adjustment on the z axis. Views of the coil holder 200 according to certain embodiments described herein are shown, for example, in FIGS. 7-8 and 36-38.

Moving the subject 400 from the supine to prone position has an unintended effect on a subject's performance. The prone position does not allow the same leverage as the supine position when attempting to move the actuating bar 110. The force resisting the extension of the distal leg also acts to raise the proximal leg and hips during the process. Subjects reported having to tighten muscles in the upper leg, hips and back to get leverage to lower the actuating bar 110.

To eliminate this problem, an adjustable brace (not shown) and pad 120 which presses on an area superior to the popliteal fossa was added to the apparatus 100. The brace added the leverage that a subject 400 needed to perform the desired movement. The brace also helped to restrict leg movement to a single axis of rotation. The brace had the effect of forcing the thigh to maintain positive contact with the surface coil 300, thus controlling motion artifacts. This embodiment is illustrated in FIGS. 6 and 11.

The apparatus 100 can be configured for use with either leg. A person of skill in the art will understand how to modify the apparatus 100 based on the desired leg to be used. The resistance was provided by a single 24 inch elastomer band, which was routed within the structure of the apparatus 100. Early designs had the elastomer band exposed. Although the internal placement of the band was considerably more difficult to assemble, the apparatus 100 shields the high tension band from the subject 400, and reduces risk of damage during use and storage. In another embodiment, an apparatus 100 was designed to provide resistance in the correct vector in the prone position apparatus 100 by including a “tower” and roller bearing. The tower is slightly offset to the left to allow the right leg and coil 300 to be better centered in the bore 150.

The pivot point 160 of the actuating bar 110 was designed with a slip bearing to withstand torque in two axes and provide a smoother operation. To eliminate heating and softening of the PVC components, thick silicon grease was applied to the load-bearing surfaces.

The base of the apparatus 100 rests on the metal portion of the couch 170 where the pads 120, 122 would normally be set so the pivot point 160 is lower than the knee joint. This is important for matching the arc of the actuating bar 110 to the non-circular arc of the knee. Moving the pivot 160 lower also slightly increased the travel distance of the bar, where space was very limited inside the bore 150.

Cylindrical pads 120, 122, as used on commercial exercise machines, were purchased and fit to the lift point. The larger diameter and thickness of the pad 120, 122 spread the load over a wider area.

Fatigue of the VL m. was reported by all subjects and the expected results were seen in the spectra collected. With the coil 300 in tight contact with the thigh and held firmly in a custom made coil holder 200, the SNR and quality of the ³¹P spectra were observed to be better in most cases.

In another exemplary embodiment, the footprint of the apparatus 100 was made smaller. The PVC used for the framework proved to be rigid enough to support the workload placed on it. The size of the apparatus 100 was reduced by 16% and the weight was reduced by 13% to 10.3 pounds (4.68 kg). The apparatus 100 was also designed to better position the VL m. and surface coil 300 in the center of the bore 150. The upper leg and patella rests horizontally on the couch 170 with the surface coil 300 trapped against the VL m. Positioning a subject 400 to the left as far as possible, the leg and coil were as close to isocenter as possible. The coil is nearly orthogonal to the scanner's native axes.

In another exemplary embodiment, pressure points are eliminated. While the scout image is acquired and slice selection is taking place, the ankle has some pressure on it from the resistive band. The foot is forced up against the scanner bore 150 and rests against the actuating arm until the exercise phase of the protocol is being performed. There is no pressure on the knee joint.

In another exemplary embodiment, the source of resistance is completely contained and hidden within the apparatus 100. There are no pinch points or any way to come in contact with the elastomer which is under heavy stress even when the apparatus 100 is not in use.

In another exemplary embodiment, the surface coil 300 is integrated into the design of the apparatus 100 to consistently position the coil 300 in the same location with respect to the isocenter and hold it in a fixed position throughout the protocol. With the coil 300 in a fixed position, the subject 400 may rest his upper leg against the coil 300.

With the subject 400 positioned prone and head first, the exercise apparatus 100 is just inside the opening of the bore 150. The chance of the apparatus 100 causing any inhomogeneity in the B₀ field is minimized.

In an exemplary aspect of the present disclosure, preparing the MRS protocol is a two-step process: (a) position and attach the exercise apparatus 100 to the scanner couch 170 before the subject 400 arrives; and (b) position the subject 400 on the exercise apparatus 100 and surface coil 300.

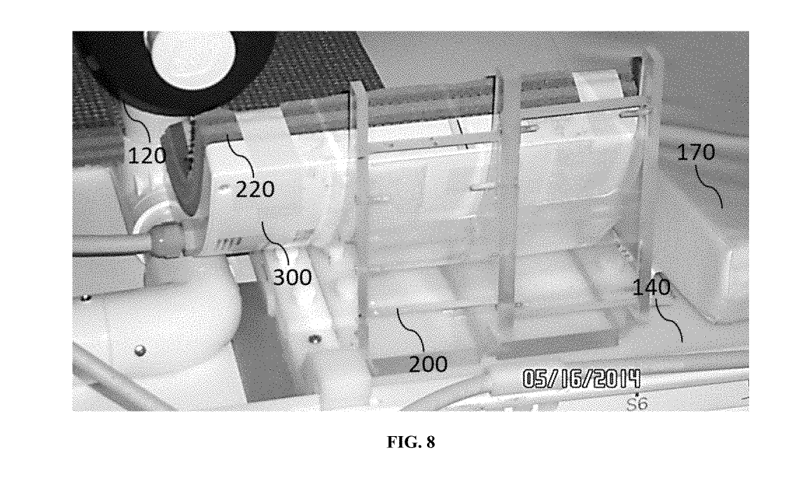

The exercise apparatus 100 has extensions, which are placed into slots on the MRI scanner table 140 that were labeled by the number of the spine coil element (S1-S8) that would be positioned nearby, as shown for example in FIGS. 8 and 9. The attached points 130 of the apparatus 100 may be placed into slots just beyond number S8 as shown in FIG. 9, because this determines the location of the surface coil 300 on the table. S7 (not shown) is the maximum position of the coil holder 200 to insure the surface coil 300 can be positioned at the magnet's isocenter. Once the brackets are fully engaged the apparatus 100 will not move at all. If the apparatus 100 does move, then the positioning process may be restarted.

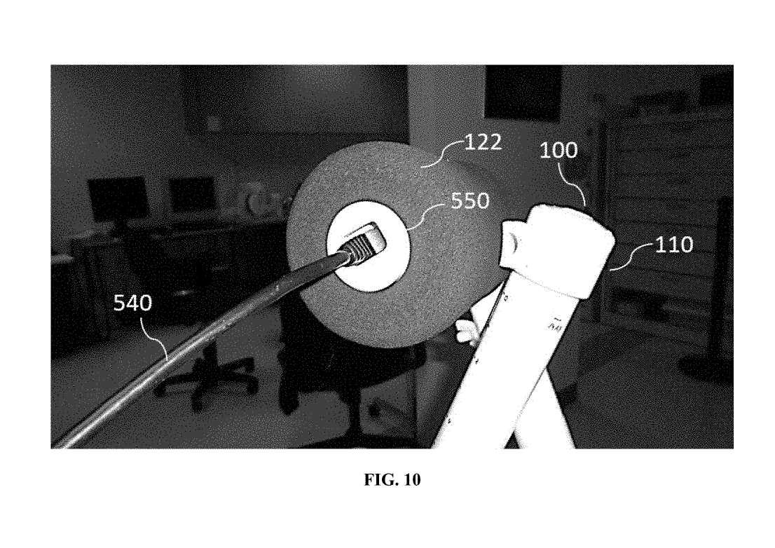

The coil holder 200 is positioned into the next available slots on the couch 170, and the surface coil 300 is positioned as shown in FIG. 11. The Ethernet cable 540 is locked into the RJ45 socket 550 found at the tip of the actuating bar 110, as shown in FIG. 10.

The subject 400 is positioned on the couch 170 prone and head first as shown in FIGS. 6, 9, 11-12, ad 22. It may be a slight challenge to get into position on the scanner couch 170 for even a person with a normal BMI. It is anticipated the positioning of some obese or mobile impaired subjects could be challenging.

The correct position of the thigh on the coil with regards to the VL m. may be verified. The subject 400 may move + or − on the Z axis to the proper position. The knee may be nearly aligned with the pivot point 160 of the actuating arm.

The leg brace may be adjusted to firmly apply pressure to the back of the upper leg and lock it in position using the nylon screw and wing nut. Care should be taken as the subject 400 is being inserted into the bore 150. The subject's foot and the actuating bar 110 may be slightly lowered as the couch 170 moves into or out of the scanner bore 150.

Running the entire MRS protocol requires about one hour, as shown in Table 1. A reference sample of methylenediphosphonic acid (MDP) (CH₆O₆P₂) in a small plastic vial is permanently attached to the face of the surface coil 300. A pad 220, attached to the coil, protects the reference sample and places it very slightly above the subject's thigh. The reference sample is important for post processing and calculating the absolute concentrations of PCr in vivo.

TABLE 1
³¹P-MRS Protocol Sequence

| | | Scan Time |
|---|---|---|
| | Scan Sequence Description | (minutes) |
| Scan 1 | Trfi 2Slice Localizer FOV 300 centered | 1:03 |
| Scan 2 | T1 Fl2d fs Coronal 256 whisper | 1:28 |
| Scan 3 | T1 Fl2d fs Axial 256 30 Slice whisper | 1:39 |
| Scan 4 | Water Reference 30 with 8 averages | 0:36 |
| Scan 5 | Water Suppression 30 with 32 averages | 1:48 |
| Scan 6 | Test Exercise; 5 measurements | 0:30 |
| Scan 7 | ³¹P-MRS TR = 500 64 averages 4 prep Flip | 0:34 |
| | angle = 90 | |
| Scan 8 | ³¹P-MRS TR = 1000 32 averages 4 prep Flip | 0:36 |
| | angle = 90 | |
| Scan 9 | ³¹P-MRS TR = 3000 16 averages 4 prep Flip | 1:00 |
| | angle = 90 | |
| Scan 10 | ³¹P-MRS TR = 6000 16 averages 4 prep Flip | 2:00 |
| | angle = 90 | |
| Scan 12 | ³¹P-MRS TR = 10,000 16 averages 4 prep Flip | 3:20 |
| | angle = 90 | |
| Scan 13 | MDP reference ³¹P-MRS TR = 10,000 16 | 3:20 |
| | averages 4 prep 90 degree flip angle | |
| Scan 14 | Slab image 5 sec st = 3 df = 108 | 1:28 |
| Scan 15 | Exercise event ³¹P-MRS 2 averages Flip | 15:00 |
| | angle = 140 Acquisitions = 150 | |
| Scan 16 | Water Reference 30 with 8 averages | 0:36 |
| Scan 17 | Water Suppression 30 with 32 averages | 1:48 |

A localizer image is obtained first to identify the muscle in which the MRS ROI is to be positioned. Water saturation and suppression scans are obtained next. Shimming is then performed. Achieving a water peak full width half maximum (FWHM) between 22 Hz and 30 Hz is desired. The operator usually records the center frequency, FWHM and slab position for later reference and verification. A test MRS scan is then performed to obtain a sample spectra used to verify that the PCr peak is centered at 0 ppm and produced with adequate signal-to-noise ratio.

For the MR spectroscopy scans, a flip angle of 90° and a TR of 3000 ms were found to provide the best SNR. The long TR allows adequate relaxation period between spectroscopy samples. The RX bandwidth is 2200 Hz and two samples are averaged per spectra (NSA=2).

The ³¹P-MRS spectral data are acquired during a 15 minute repeated acquisition sequence. The subject 400 is informed shortly before the exercise period starts. The exercise sequence is started with a 2 minute baseline period during which the subject 400 remains still. The subject 400 is then told to start moving the actuating bar 110 fully down and return it to the top position within about one second. The exercise bout lasts for approximately 5 minutes during which approximately 50 spectra samples are acquired. During this period, PCr levels may begin to drop while Pi levels increase. Also during exercise, a software program emulating a strip recorder is activated on the strain gauge 520 signal processing computer to capture the amount of force the subject 400 is using to move the actuating bar 110. The strip recorder simultaneously monitors the position of the bar in the arc of its movement. These two data sets allow for the calculation of the approximate total amount of energy, in Joules, that a subject 400 imparts to the exercise apparatus 100. A second measurement of total Newton·seconds is also available, indicating just the force applied by the subject 400. These data are processed after the scan.

The subject 400 is asked to move in between spectra samples and to attempt to be stationary when the sample is acquired. Subjects 400 can time their motion based on the clicks heard inside the scanner caused by the switching of the gradient magnets. This coordination is desirable, but not critical to acquire good quality spectra.

At the end of the five minute exercise period the subject 400 is told to stop exercising for an eight minute recovery period. During this time, 80 spectra samples are acquired which may show PCr levels rising to nearly pre-exercise levels while Pi levels are falling simultaneously.

A final water saturation and suppression measurement is taken before the subject 400 is removed from the scanner.

After the subject 400 has been released, it has been a practice to place a 1000 mL phantom with 35 mMol concentration of phosphoric acid (H₃PO₄) in place of the thigh and perform an MRS scan to be used as a reference for processing the spectral data. Care is taken when removing the subject 400 so that the coil does not move and the phantom is placed on the surface coil 300 with the MDP still in position.

This completes the scan sequence.

³¹P-MRS scan data are stored on the Siemens 3T Magnetom Trio MRI scanner as a proprietary data file known as raw data (.rda). Each spectrum, which consists of the average of two data acquisitions, is stored on the scanner hard disk drive in a sequential filename. At the end of the protocol the .rda files are copied to external storage for archive and post processing. Since the file is proprietary not much is known of its structure.

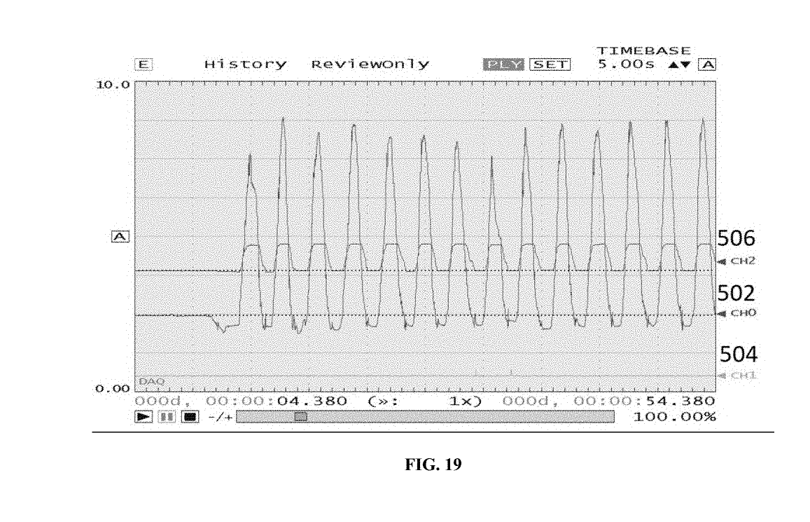

While at the control console, the patient browser can examine spectra either during the protocol or after the protocol is completed. Spectra appear on the monitor, as seen in FIG. 19. This sample is a baseline spectrum where the PCr signal is shown as a tall spike at a chemical shift of 0.0 ppm and the Pi is a much smaller spike at a chemical shift at about 5.0 ppm when the PCr peak is centered at 0.0 ppm. Fitting of known chemical shifts is built into the scanner software. Also seen on this sample are the three spectral peaks produced by ATP (α,β,γ).

After a period of exercise the VL m. PCr level may have decreased substantially with a concurrent rise in the Pi signal. During the recovery time the PCr levels may return to near the earlier baseline level. As ATP is created in the muscle cells, Pi is consumed. The rate of recovery (k) is a target parameter. Caution must be taken when trying to interpret the MRS spectra on the system monitor. The scale of the signal spikes is auto-ranging so that two spectra may look similar, but are of different amplitudes due to the scale of the y-axis. Post processing with jMRUI removes the ambiguities due to the auto-ranging of the MRI system MRS display.

For processing the MRS raw data files (.rda) the Java based jMRUI and RStudio statistical software packages were chosen. The graphical user interface of jMRUI integrates a number of programs to perform either time domain or frequency domain analysis of in-vivo spectra data sets. For the ³¹P-MRS, time domain analyses were performed on FID signals. Prior knowledge of the spectra was included to allow the software to better quantify peaks around the PCr parameters.

The extracted 150 .rda files are placed in one folder on a computer. jMRUI is opened and directed to that folder to load the files. The jMRUI program displays the 150 individual spectra referenced to the same scale. The spectra are now ready for preprocessing. The spectra are apodized with a 2.5 Hz Lorentian function to enhance the peak of the spectral while reducing side lobe and below the x-axis signal noise. The spectra are next subjected to phase correction so that the PCr peak is just above zero (about 50 to 65 degrees). A zero reference is set on a peak and then the AMARES algorithm is used to quantify the spectra. Starting values and “prior knowledge” are loaded into jMRUI to help the software produce useful results. The results are saved as an Excel text file.

The Excel file is opened and the amplitudes of the PCr and Pi spectra are found and plotted with a time column added. Data after t=420 (before start of exercise) is saved as a tab delimited text file for statistical analysis.

RStudio is used with the NLRWR library to do the final processing using the equation:

nls(PCr˜480+D*(1−exp(−k*time)))

The last line of the statistical results is the final value of D and k, where k is the rate time constant we are trying to measure. Recovery times averaged between 40 and 60 seconds depending on the individual. Values of k have been observed to be between 0.0120 and 0.0539. The value of k is an indirect indication of ATP production and mitochondrial function, or impairment as in insulin resistance subjects.

Values for T1 are estimated using the spectral data from scans 7 to 12. These scans are identical except that the TR time varies from 0.5 to 10 seconds. These same spectra data sets can also produce an indication of the pH drop that occurs in skeleton muscles during exercise.

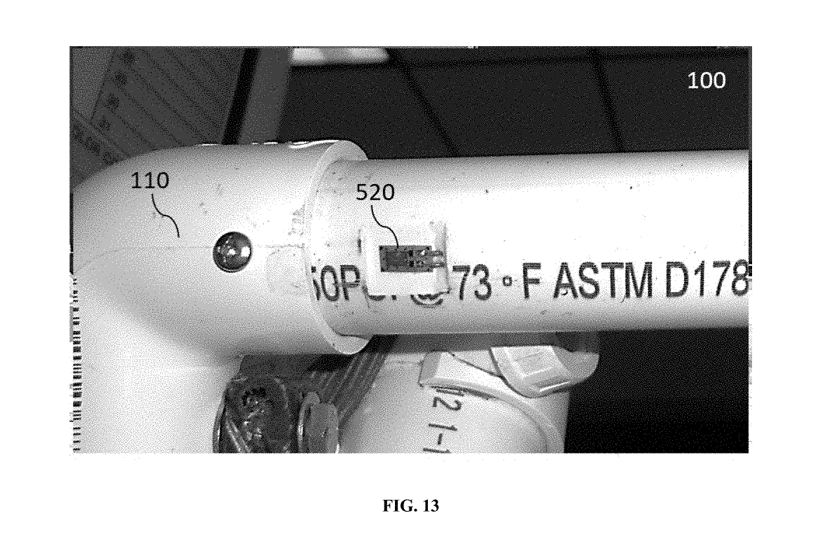

An exemplary embodiment of the exercise apparatus 100 of the present disclosure was the inclusion of instrumentation 500 to capture the work output of each subject 400 during the exercise period. In order to provide this feature, strain gauges 520 were employed. Referring to FIG. 13, the strain gauges 520 were cemented to surfaces of the actuating bar 110, which had been machined perfectly flat. Since a strain gauge 520 is highly directional in a single direction, the two machined surfaces had to be opposed by exactly 180 degrees to minimize error.

Another exemplary embodiment of the circuit 530 is the cement used to attach the strain gauge 520 to the PVC structure. The glue had to permanently adhere to PVC and provide a durable bond. The cement had to dry hard so that the strain gauge 520 experiences the full stress that is placed on its structure. If the cement is even slightly elastic, measurement errors will be encountered and the bond will eventually fail.

Although strain gauge 520 manufacturers recommend a number of cements, none are recommended for PVC adhesions and most are expensive. Manufacturers recommend against attaching strain gauges 520 to plastics for a number of reasons, so help was unavailable in selecting a suitable cement for this application. A period of trial and error ensued while attempting to find the right cement. The selected cement is an isocyanate prepared polymer with urethane links and has performed better than expected.

An outer PVC cover (not shown) fits over the strain gauge 520 installation to protect it from physical abuse. 30 gauge wires were soldered to the strain gauge 520 and routed into the cavity of the bar though tiny holes which supported and separated the wires. A cylindrical pad 122 slips over the PVC cover and contacts the foot. The instrumentation amplifier 510 and Cath RJ45 connecter 550 fits inside the actuating bar 110, making the entire instrument a small, self-contained unit.

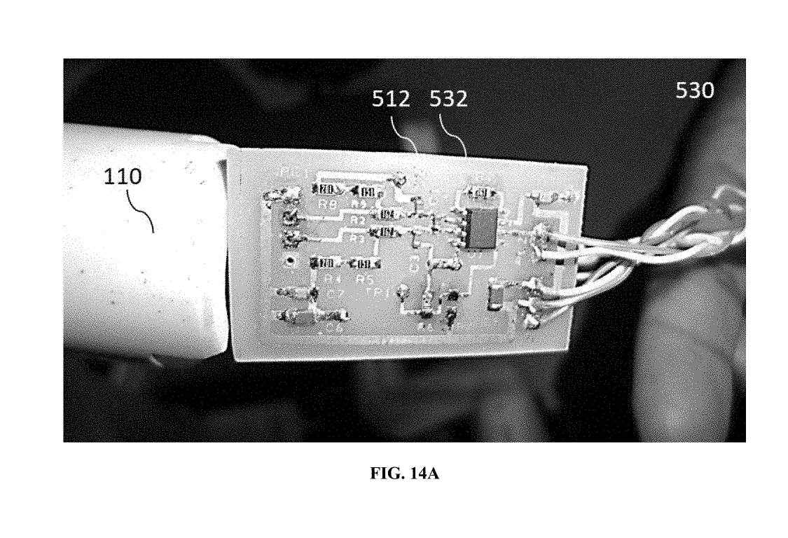

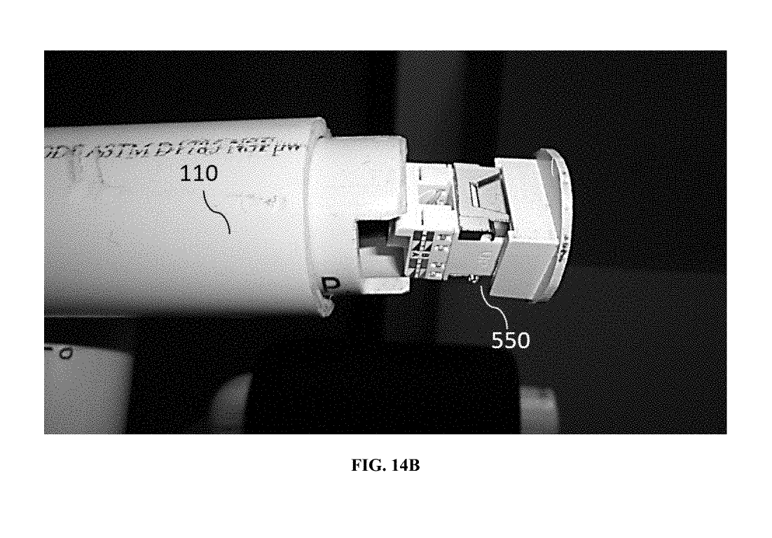

Referring to FIG. 14A, the instrumentation amplifier 510 was assembled on a custom PCB circuit board, designed and etched in-house. Surface mount devices (SMD) were used to minimize the size of the circuit board allowing placement within the cavity of the actuating bar 110 (1.0×7.0 inches). An Analog Devices “High Performance, Low Power, Rail-to-Rail Precision Instrumentation Amplifier” (AD8422) was chosen to simplify the circuit and power requirements. The circuit board is 4.20 cm by 2.54 cm, double sided etched from 1 oz. copper clad FR-4 glass-epoxy material with a ground plane on bottom.

+9 volts DC power was supplied to the circuit 530 from the control room via a single 4 pair, Cat7 twisted pair data cable 540. Analog data, power and a single point ground, and a control circuit entered the amplifier 510 via a grounded Cath RJ45 data connector 550 built into the actuating bar 110. The circuit board supplied filtered power to the strain gauge circuit 530 which will be described in a following section.

Due to the environment in which the strain gauge circuit 530 had to perform, much thought went into the design and components selected. Because of the strong magnet field and high slew rate of the gradients, 12 mil wide circuit traces were used along with small 0805 SMD resistors and capacitors (2.0 mm×1.25 mm, 0.5% tolerance, or better) and a single small outline integrated circuit (SOIC), and a solid ground plane. The tiny components and short traces minimized transient circuit noise which could be caused by the normal operation of the MR scanner. The ground plane is a good design practice for noisy environments where a single point ground reference is required as in the case of analog signals.

The Cat7 cable 540 was selected because it is an engineered cable with an 80% tinned copper braided shield. It was discovered RFI was being coupled into the cable from the control room and added a discernable amount of noise to the spectra data. The data cable 540 is grounded at the bulkhead where the cable enters the scanner room. The four (4) twisted pair conductors have been engineered for common mode noise rejection with a small solid gauge wire. Cat7 cable 540 has a durable jacket and readily available connector components so the cable 540 to the control room could be quickly and reliably connected and removed from the exercise apparatus 100.

The concept of strain is based on Hooke's law of elasticity. When an external force is placed on a solid object, an internal force develops in relation to the cross sectional area of the object. The object is said to be under stress (σ) which is measured in N/m², following the equation:

$$σ = \frac{P}{A}$$

where P is the external force applied and A is the cross sectional area.

If a longitudinal force is applied, the object elongates or compresses by ΔL which could be large or minute. The ratio of elongation or compression to the original length, L, is referred to as “strain” which is dimensionless, but may be given in parts per million.

$${ɛ}_{1} = \frac{Δ L}{L}$$

When an object is stressed and ΔL occurs, the diameter or thickness of the object is reduced by a proportional amount as long as the material remains in its elastic phase and has not entered into the plastic phase.

$${ɛ}_{2} = \frac{- Δ d}{d}$$

The ratio of longitudinal strain (ε₁) verses lateral strain (ε₂) is called the Poisson's ratio (υ):

$$υ = \vert \frac{{ɛ}_{2}}{{ɛ}_{1}} \vert$$

Where there is a linear relationship between stress and strain, i.e., where Hooke's law of elasticity exists (elasticity vs plastic), a proportional constant, E, also exists, which is known as Young's modulus or the modulus of elasticity. Values of E depend on the material and have been determined experimentally in most materials.

Strain (μm/m) is given by:

$$ɛ = \frac{σ}{E}$$

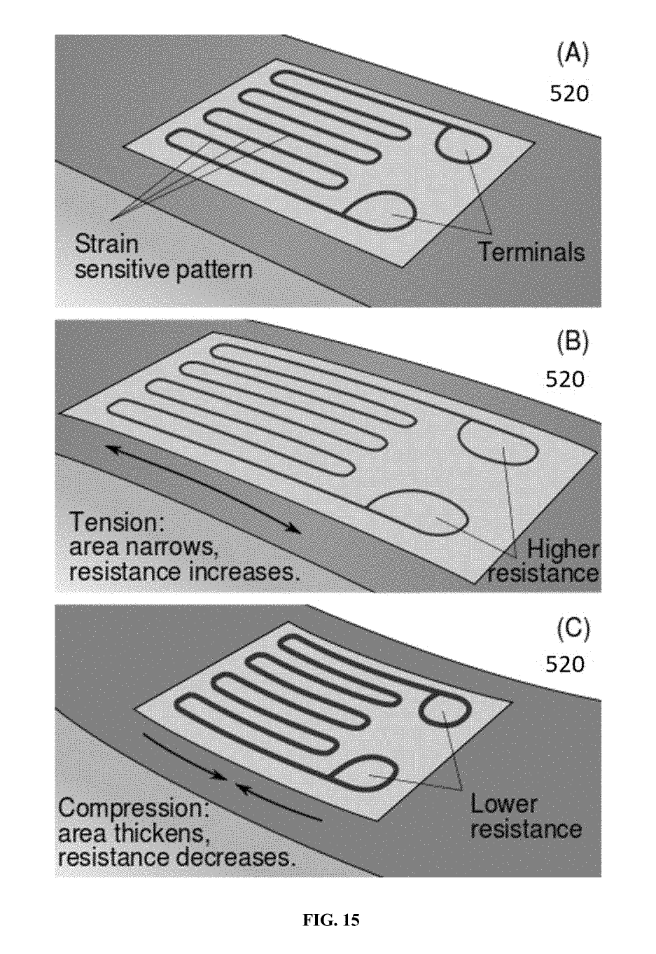

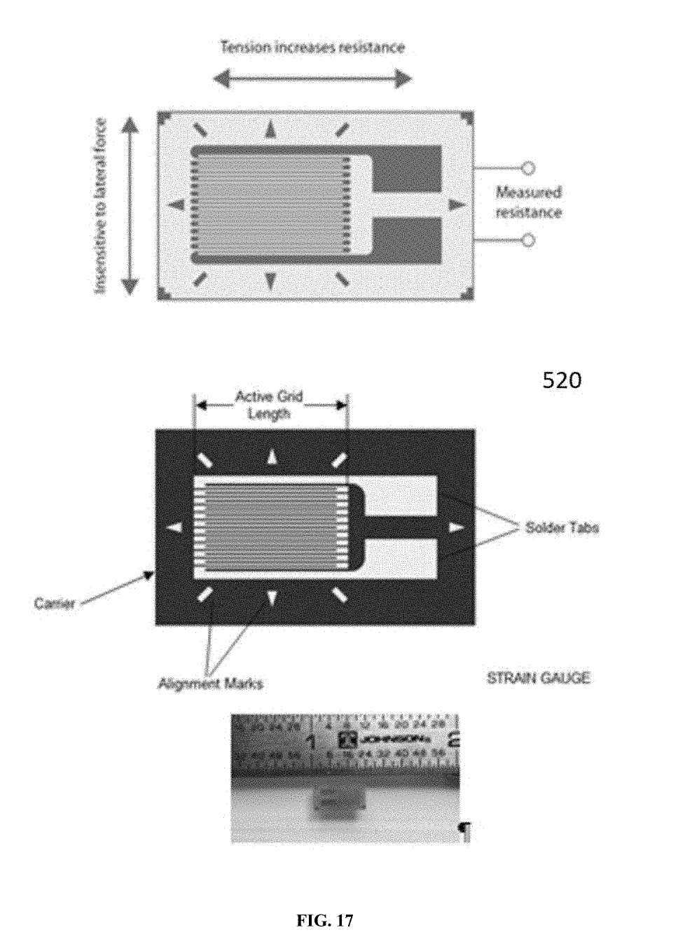

Referring to FIGS. 15 and 17, a strain gauge 520 is a tiny copper foil electrical circuit which is very tightly bonded to a thin plastic film. Its electrical resistance changes slightly due to changes in conductor cross-section and length when the gauge is experiencing strain. The copper foil sensing element is actually an alloy such as copper-nickel which has a well-defined inherent resistance. Most conductive metals will exhibit a slight change in resistance when elongated or shortened. A strain gauge 520 uses this minute change in resistance to measure strain in a material. FIG. 17 illustrates the mechanical concept of a strain gauge 520. Every strain gauge 520 has a proportional constant called a gauge factor (G.F. or K) specified by the manufacturer. A copper-nickel strain gauge 520 has a K=2. When a strain gauge 520 is tightly bonded to a material to be monitored, the foil circuit is able to convert mechanical strain (ε) into a corresponding change in resistance (ΔR) accord to:

$$\frac{Δ R}{R} = K * ɛ or Strain = \frac{( \frac{Δ R}{R} )}{GF}$$

Even under heavy strain the AR may be less than 0.2%.

An exemplary embodiment of the apparatus 100 of the present disclosure has a strain gauge 520 having an R=350Ω. With the relative small strain placed on the actuating bar 110, ΔR would be around 0.3Ω which would be extremely hard to measure. Fortunately an accurate resistance measurement is not required.

The resistive type strain gauges 520 are sensitive to temperature variations. Ohm's Law states, E=I*R. Since the coil foil is slightly resistive in nature and an external voltage is applied to the gauge, a small amount of heat is generated by the current flowing through the foil according to P=I*E or P=I²*R. This generated heat changes the resistance of the foil slightly by Brownian motion, causing a tiny error in ΔR which is sensed, amplified, and appears at the output of the instrumentation amplifier 510. The circuit 530 had to be self-correcting in order to compensate for temperature variations.

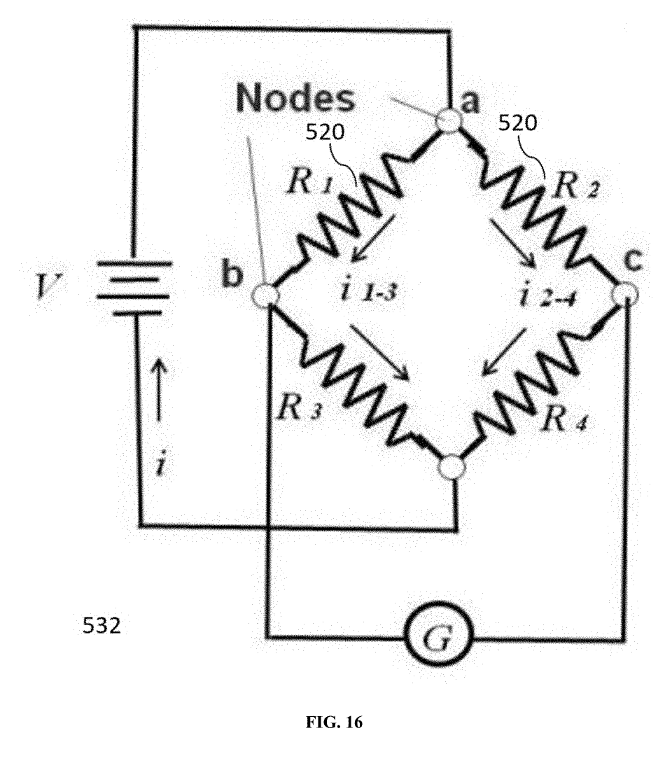

The strain gauge circuit 530 consists of four parts, as shown in FIG. 16. Two strain devices 520 are cemented to the actuating bar 110 separated by 180±2 degrees. The devices 520 form part of a Wheatstone bridge 532 circuit along with two additional precision resistors. The devices 520 are positioned so they sense strain in only one axis from torque in the bar. That axis has been positioned to face the force applied by the foot. Since the bar is a cantilevered beam, the upper gauge elongates and the lower gauge compresses when torque occurs.

A Wheatstone bridge network 532 consists of four closely matched resistors as shown in FIG. 16. R1 and R2 have been replaced by the strain gauge 520 components, where the resistance changes minutely. With zero strain and assuming all resistor values are identical, the current through R1, R3 is identical to the current through R2, R4 as derived from Ohm's law.

$${i}_{1 , 3} = \frac{V}{{R}_{1} + {R}_{3}} and {i}_{2 , 4} = \frac{V}{{R}_{2} + {R}_{4}}$$

The voltages at node b and node c are identical, so the output G will equal zero.

$${V}_{b} = \frac{V * {R}_{3}}{{R}_{1} + {R}_{3}} and {V}_{c} = \frac{V * {R}_{4}}{{R}_{1} + {R}_{4}}$$

$$G = {V}_{b} - {V}_{c}$$

Output G is actually the filtered input of the very high impedance instrumentation amplifier 510. The high impedance of the amplifier 510 prevents loading the bridge circuit 532 and skewing the output.

In an exemplary embodiment of the present disclosure, when increased tension is detected by R1 and increased compression in R2, then R1 resistance increases and R2 resistance decreases. Thus current i_{1,3} decreases and current i_{2,4} increases by a similar amount. The output G increases by about 5 to 10 mV as V_{b} increases and V_{c} decreases. The amplifier 510 with a fixed gain of 500 raises the output of the circuit 530 to a measurable level. The exact values of V_{b} and V_{c} are irrelevant.

The placement of R2 is important because it also serves as a temperature compensator in the bridge. This is accomplished by selecting the correct leg of the bridge 532 for the second component. A strain gauge 520 has a positive temperature coefficient; meaning the resistance increases with temperature. If R2 was a fixed resistor instead of a match to R1, two different coefficients would exist in the left and right side of the bridge 532 and it would always be unbalanced with a non-linear offset over temperature. Since both devices 520 are at the same temperature, the balance of the bridge 532 will remain constant.

Referring to FIG. 18, many strain gauge 520 designs employ a low pass filter 512 between the Wheatstone bridge 532 and the instrumentation amplifier 510. The filter 512 reduces ripple and noise generated in the strain device and the connecting wires. The length and quality of the connecting wires can become a design concern. Referring to FIGS. 14A and B, a common mode rejection low pass filter 512 was included in the design since the results of operating the strain gauges 520 and amplifier 510 in a moving structure within the B₀ field was a large operational risk. There was also the risk of electromagnetic interference (EMI) and radio frequency interference (RFI) from the TX/RX coil and the gradient magnetic fields. Bypass capacitors are placed across Vss and GND to remove ripple in the power source. With a gain of 500, there was also a high risk of the amplifier 510 spontaneously oscillating.

Still referring to FIGS. 14A and B, and 18, in an exemplary embodiment of the present disclosure, a custom built interface device was designed to easily and quickly connect the exercise apparatus 100 to the data acquisition unit (DAQ) 514. Designed as a small cube, a grounded Cat6 RJ45 connector 550 accepts the Cat7 cable 540 from instrumentation circuit 530 and a second RJ45 connector passes analog signals to the DAQ 514.

The interface accepts regulated +9.0 VDC from a 120 VAC power converter and applies the Vss supply voltage to the Cat7 cable 540 per Power over Ethernet (PoE) specifications. The interface also houses an adjustable voltage follower op-amp circuit to supply a low impedance reference voltage to the instrumentation amplifier IC to bias the output signal.

In an exemplary embodiment, a Measurement Computing Corporation (MCC), 8 channels DAQ was purchased to convert the analog signals to digital. The USB-201 is a 12-bit single ended analog to digital converter (ADC) with a maximum sampling rate of 100 k samples/sec. The DAQ interfaces to an ordinary USB port on a computer. A rate of 50 samples/sec (period=20 ms) supplied ample data. The DAQ accepts analog inputs of ±10 V, but to simplify the power requirements, a range of 0 to +9.0V is expected at the DAQ.

Referring to FIG. 19, three channels are monitored and recorded by the DAQ 514. Channel 0 502 accepts an analog voltage from the instrumentation amplifier 510 as an indication of the amount of force (Newtons) a subject 400 is placing on the actuating bar 110. Higher force is seen with harder exercise which in turn causes a higher voltage to appear at the DAQ 514. Channel 1 504 monitors and records the output of the reference voltage op-amp in the interface cube, which is set before use and may remain constant. Channel 2 506 monitors and records a voltage corresponding to the position of the actuating bar 110 in its arc from the full up to full down position. A potentiometer mounted internal to the exercise apparatus 100 follows the movement as the bar is actuated. Higher voltage is seen at the DAQ 514 input as leg extensions move the actuating bar 110 down.

MCC provides a simple-to-learn and easy-to-use Windows-based program which emulates a strip recorder. The program graphically presents the analog signals in real time, as well as capturing numerical values from the ADC and storing them in a .cvs spreadsheet file.

Setting the strip recorder to 50 Hz captures a data sample every 20 ms. The recorder is activated in the control room just before the five minute exercise period begins. At the end of exercise, the .cvs file contains 16,000 data points indicating the subject's work output. The data is post processed to determine the amount of effort the subject 400 imparted to the exercise apparatus 100.

FIG. 19 shows results from an exercise period in graphical form produced by the strip recorder.

Before exercise begins, baseline measurements are recorded from each channel. Channel 0 502 represents the amount of force a subject's foot is placing on the articulating bar during the VL m. extension motion. Since the resistance is a tension which is nearly linear over the range of motion, one could equate the elastic tension to lifting weight (i.e. mass) and correctly assume that a larger force produces a higher acceleration of the leg (F=ma). Since the range of motion is limited, a subject 400 will reach the endpoint in less time.

Using applied force (strain) from the baseline value to the peak value vs time for each downward leg extension and following leg flexion, a summation of all the data points will produce a number relative to the amount of work or energy imparted to the apparatus 100. Looking at channel 0 502, the area under its curve is the energy expended. As an example, with less force on the actuating bar 110 there would be a lower leg velocity, but a longer time to reach full extension and the graph would appear triangular with a low height and a wide base. A quick movement with high force would appear more spiked with a higher amplitude and faster rise time. Should a subject 400 choose to hold the bar down for a short time (e.g. 1 second); they would still be applying force to hold their position and expending energy during that time.

Through empirical measurements using an electronic scale, the strain gauge circuit 530 has been calibrated (in kg) to the elastic tension (weight) of the resistance band. The circuit 530 produces its maximum value of 8.95 VDC with approximately 17.5 kg (159 N) of force applied to the actuating bar 110.

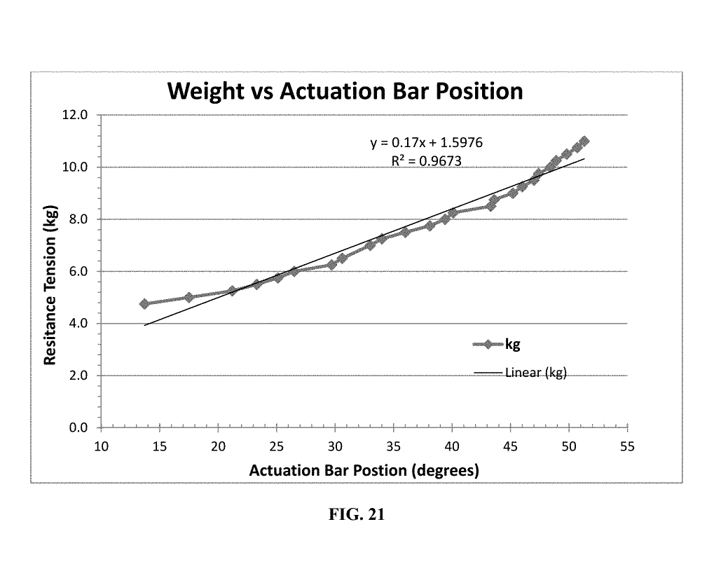

Since the bar is moving and accelerates, Newton's second law is used to relate the applied force and resistive tension. As the bar moves from the full up to full down position, the elastic resistive tension increases per Hooke's Law minus some small un-measurable frictional forces. This motion produces a slightly non-linear graph of position vs tension, as shown in FIG. 21. At full extension, the elastomeric band provides about 11 kg (108 N) of resistance. The position indicator data provides information to determine the instantaneous velocity of the foot (bar) per each 20 ms.

TABLE 2
Strain Gauge and Position Indicator Calibration Data

| | Position | Resistance | | |
|---|---|---|---|---|
| Degrees of | Indicator | Tension | Strain Gauge Output | |
| Movement | (volts) | (kg) | (volts, absolute) | Kg - Force |
| 0 | 3.000 | ND | 2.40 | ND |
| 0.5 | 3.100 | 1.27 | 2.75 | 1.17 |
| 3 | 3.200 | 3.83 | 3.00 | 1.74 |
| 5 | 3.300 | 4.14 | 3.25 | 2.70 |
| 11 | 3.400 | 4.55 | 3.50 | 3.24 |
| 20 | 3.500 | 5.04 | 3.75 | 3.83 |
| 24 | 3.600 | 5.53 | 4.00 | 4.36 |
| 29 | 3.700 | 6.40 | 4.25 | 4.89 |
| 34 | 3.800 | 7.21 | 4.50 | 5.69 |
| 39 | 3.900 | 7.85 | 4.75 | 6.29 |
| 44 | 4.000 | 8.74 | 5.00 | 6.90 |
| 50 | 4.100 | 10.22 | 5.25 | 8.12 |
| 52 | 4.160 | 11.00 | 5.50 | 9.09 |
| | | | 5.75 | 9.75 |
| | | | 6.00 | 10.48 |
| | | | 6.25 | 10.93 |
| | | | 6.50 | 11.40 |
| | | | 6.75 | 11.53 |
| | | | 7.00 | 12.19 |
| | | | 7.25 | 12.65 |
| | | | 7.50 | 13.79 |
| | | | 7.75 | 14.72 |
| | | | 8.00 | 15.23 |
| | | | 8.25 | 15.60 |
| | | | 8.50 | 16.00 |
| | | | 8.75 | 16.75 |
| | | | 8.95 | 17.50 |

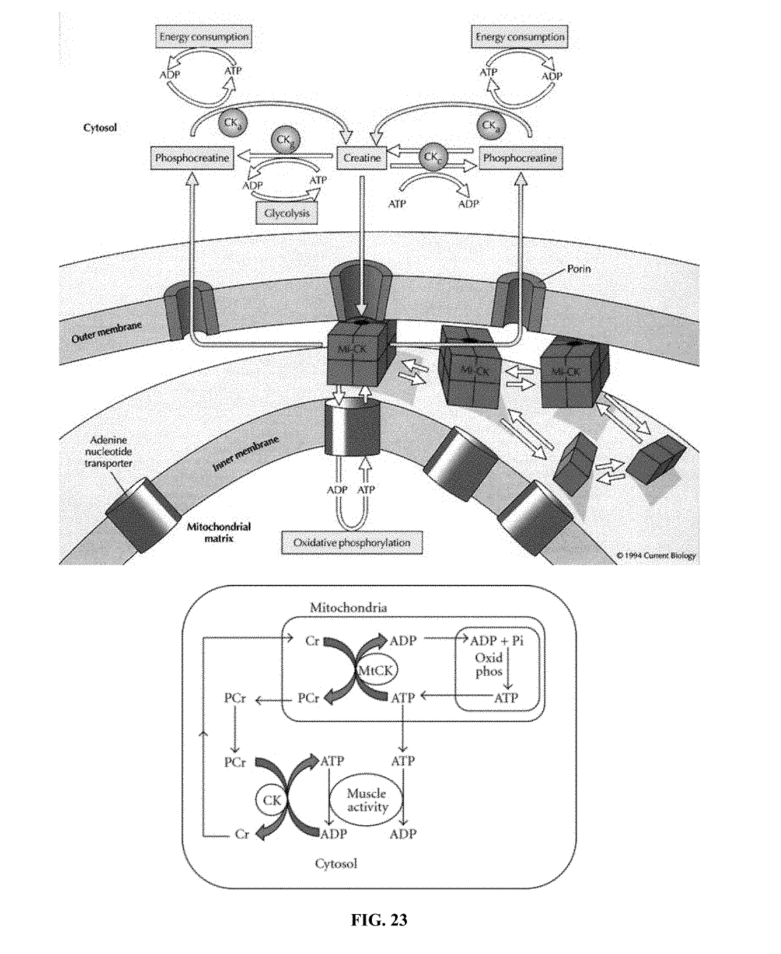

Phosphocreatine+MgADP+H+⇔MgATP2+Creatine forms the core of an energy metabolic process known as the PCr circuit, as illustrated in FIG. 23. In this circuit, the cytosol enzymes are coupled to glycolysis and produce ATP for muscle activity. The MtCK enzyme is closely coupled to the electron transport chain and can use mitochondrial ATP to generate PCr, which readily diffuses into the cytosol to resupply cytosolic PCr. This shuttle system is critical for the production and maintenance of the energy supply.

Over the previous two years, eleven subjects completed the developed exercise protocol at least two times and some three times. In order to prove the repeatability of the protocol, 2 subjects (one 25 y.o. female, and one 59 y.o. male) were scanned 6 and 10 times respectively. Processing the ³¹P-MRS and T1 data from these two subjects, it was determined that the combination of the exercise apparatus 100 and the fifteen minute exercise routine produced repeatable results for the PCr recovery rate constant, k. The maximum variation observed was 12.8% with an average variation of 8.2%.

PCr(t)=PCr(0)+D*[1−e^{(-kt)}]

Nonlinear regression is performed using this Levenberg-Marquardt equation for fitting the amplitude of recovery, D, and the recovery time constant, k. The value PCr(t) is approximately equal to the baseline value of PCr, which is also the equilibrium value of PCr after a full recovery period. PCr(0) is the value of PCr at the end of the exercise period which is presumed to be the minimum value. The value of t is the recovery period in seconds, which is 480 seconds. These inputs are provided to RStudio to fit for the correct values of k and D. Results for PCr recovery time constant, k is shown in Table 3.

TABLE 3
Results for PCr Recovery Time Constant, k (1/sec).

| | | | PCr (t0) | | k (1/s) | | |
|---|---|---|---|---|---|---|---|
| Subject # | | PCr | End of | PCr % | (recovery | k % | |
| Age/Sex | Date | Baseline | Exercise | Drop | constant) | Different | D |
| #1 30/M | 4/15 | 2043 | 1390 | 32.0% | 0.0134 | | 653.2 |
| #1 30/M | 4/29 | 1410 | 740 | 47.5 | 0.0145 | | 670.2 |
| #1 30/M | 5/6 | 1360 | 850 | 37.5 | 0.0073 | | 515.5 |
| #1 30/M | 5/29 | 1329 | 920 | 30.8 | 0.0187 | | 408.8 |
| #1 30/M | 9/26 | 1298 | 915 | 29.5 | 0.0110 | | 383.3 |
| #2 27/M | 4/17 | 1519 | 700 | 53.9 | 0.0127 | | 819.0 |
| #2 27/M | 4/25 | 1250 | 670 | 46.4 | 0.0153 | | 579.9 |
| #2 27/M | 6/12 | 1289 | 670 | 48.0 | 0.0178 | | 618.7 |
| #3 39/M | 4/18 | 1023 | 650 | 36.5 | 0.0183 | | 373.5 |
| #3 39/M | 4/25 | 1202 | 930 | 22.6 | 0.0663 | | 271.6 |
| #3 39/M | 5/20 | 1095 | 820 | 25.1 | 0.0374 | | 274.8 |
| #3 39/M | 6/5 | 1020 | 830 | 18.6 | 0.0194 | | 189.6 |
| #4 56/M | 5/5 | 1320 | 680 | 48.5 | 0.0120 | | 640.8 |
| #4 56/M | 5/8 | 1313 | 900 | 31.5 | 0.0336 | | 413.4 |
| #4 56/M | 5/23 | 1040 | 450 | 56.7 | 0.0132 | | 590.6 |
| #5 16/M | 7/14 | 1466 | 1240 | 15.4 | 0.0203 | 12.8 | 214.7 |
| #5 16/M | 7/15 | 1474 | 1090 | 26.1 | 0.0229 | 12.8 | 307.4 |
| #6 31/F | 5/8 | 1263 | 1000 | 20.8 | 0.0137 | | 263.1 |
| #6 31/F | 6/18 | ND | ND | ND | ND | | ND |
| #7 30/F | 5/8 | 1184 | 1059 | 14.4 | 0.0306 | | 178.7 |
| #7 30/F | 5/16 | 1147 | 839 | 26.0 | 0.0539 | | 295.5 |
| #8 65/F | 5/22 | 752 | 490 | 34.8 | 0.0218 | | 261.7 |
| #8 65/F | 5/26 | 920 | 440 | 52.0% | 0.0222 | 4.8% | 418.9 |
| #8 65/F | 5/26 | 952 | 649 | 31.7% | 0.0233 | 4.8% | 310.3 |
| #9 31/F | 8/1 | 1405 | 776 | 44.8 | 0.0164 | −9.8 | 700.0 |
| #9 31/F | 8/6 | 1468 | 716 | 51.2 | 0.1701 | −9.8 | 536.8 |
| #10 59/M | 4/29 | 1117 | 330 | 70.5 | 0.0169 | | 786.9 |
| #10 59/M | 5/5 | 1052 | 420 | 60.1 | 0.0144 | | 632.0 |
| #10 59/M | 5/8 | 1448 | 350 | 75.8 | 0.0176 | | 1098.0 |
| #10 59/M | 5/16 | 916 | 361 | 60.6% | 0.0155 | | 613.8 |
| #10 59/M | 5/16 | 969 | 250 | 73.7 | 0.0169 | | 700.6 |
| #10 59/M | 5/29 | 1241 | 770 | 63.7 | 0.0209 | | 423.1 |
| #10 59/M | 6/16 | 1135 | 480 | 55.3 | 0.0136 | | 572.4 |
| #10 59/M | 7/8 | 1164 | 580 | 50.2 | 0.0144 | | 536.3 |
| #10 59/M | 8/11 | 1135 | 605 | 46.7 | 0.0170 | | 536.8 |
| #10 59/M | 9/30 | 1069 | 456 | 57.3 | 0.0131 | | 527.6 |
| #11 25/F | 5/6 | ND | 550 | 53.7 | 0.0146 | | 638.2 |
| #11 25/F | 5/29 | 1111 | 560 | 49.5 | 0.0120 | | 534.8 |
| #11 25/F | 6/11 | 1177 | 370 | 68.6 | 0.0136 | | 690.6 |
| #11 25/F | 6/16 | 1338 | 360 | 73.1 | 0.0105 | | 922.0 |
| #11 25/F | 7/23 | 1216 | 530 | 56.4 | 0.0172 | −4.8 | 606.3 |
| #11 25/F | 7/24 | 1232 | 830 | 33.4 | 0.0157 | −4.8 | 450.5 |
| #11 25/F | 8/12 | 1299 | 724 | 44.3 | 0.0167 | | 560.8 |

The nominal value fork and D is between 0.012 and 0.037, and 200 to 900, respectively. To prove the protocol was repeatable, the time constant, k, was the target variable. Data from a representative experiment are shown in FIG. 24. During exercise, the subject's ³¹P spectra showed the expected drop in the level of PCr coincident with a rise in Pi. The subject's PCr level dropped to about 50% of baseline, which was the target value for PCr reduction. The slope of the recovery was also consistent with previously reported values.

Table 3 presents a synopsis of the spectroscopy data results showing the values of k and D, plus the baseline and maximum drop of PCr at the end of exercise. The tight cluster of values fork demonstrates the repeatability of the protocol and the ability of the exercise apparatus 100 to reduce the PCr level to about 50% within five minutes of exercise. The quality of the data sets improved as the protocol and exercise machine was refined during 2014. All subjects have reported that their legs were noticeably fatigued after five minutes.

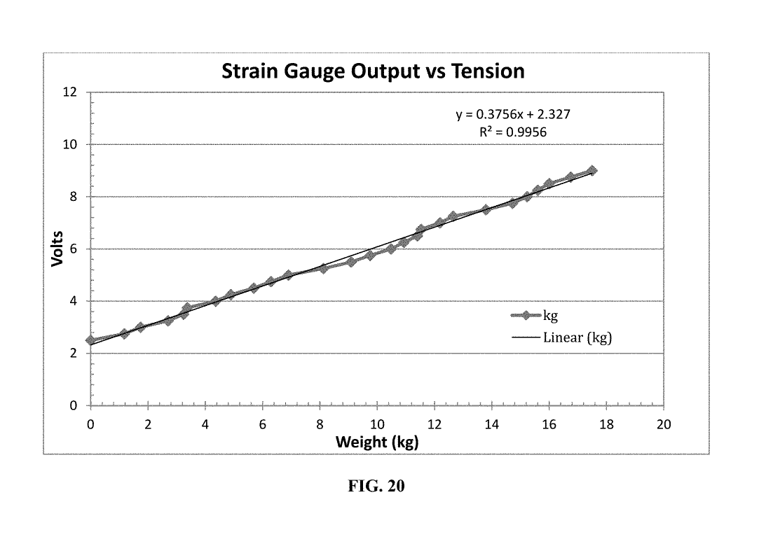

There was tight convergence of the data in the graph, shown in FIGS. 20 and 21, indicating a high SNR, which turned out to be a low noise environment during this study. Many of the scans before this showed a scatter of data points around a similar averaged line. As in FIG. 20, not to be bound by any theory, there is a positive correlation between tension (expressed in weight) and strain gauge output (expressed in voltage). As in FIG. 21, not to be bound by any theory, there is a positive correlation between actuation bar position (expressed in degrees) and weight (expressed in resistance tension).

Reproducibility of the ³¹P-MRS measurements was evaluated in five healthy volunteers (2 male/3 female, age 39.2±21.9 years) who underwent the quantitation and exercise protocols on two separate occasions. Relevant subject data are depicted in Table 4.

TABLE 4

| Parameter | Average | Range |
|---|---|---|
| Age (years) | 39.2 ± 21.9 | 16-65 |
| Sex (Males/Females) | 2/3 | |
| Weight (kg) | 70.4 ± 12.0 | 56.7-88.5 |
| Body mass index (kg/m²) | 25.1 ± 4.8 | 21.9-33.5 |

Data are presented as Mean ± Standard Deviation

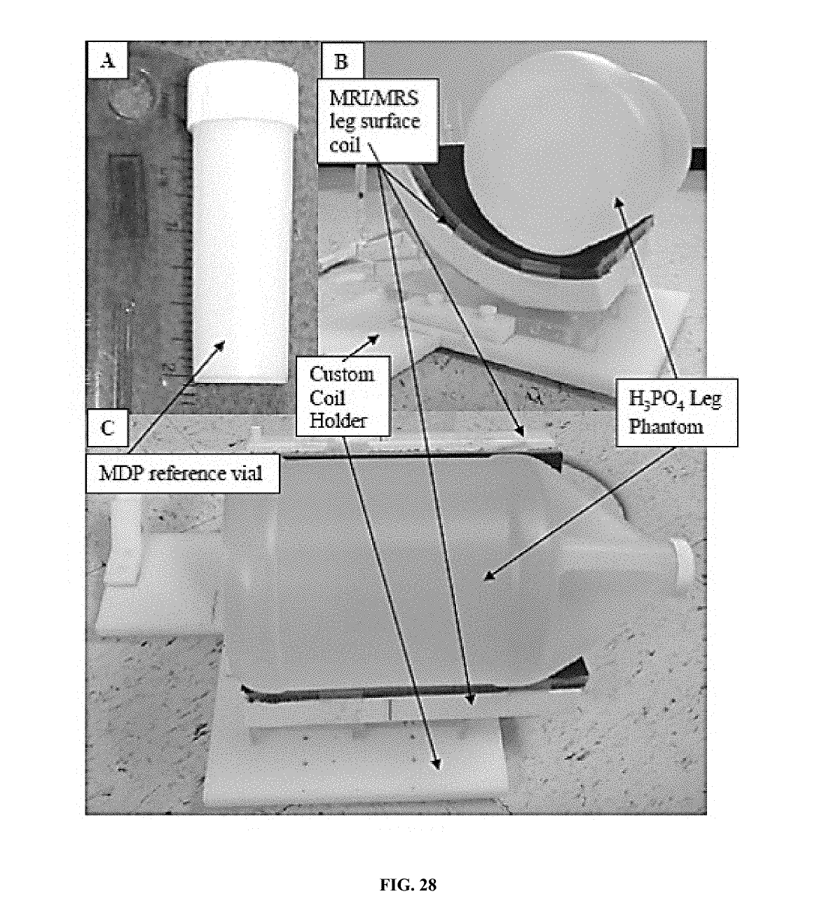

Measurements of ³¹P-MRS signal-to-noise ratios were acquired in a 3 Tesla MM system (TIM Trio, Siemens Medical, Malvern Pa.) to assess spectral quality. A ³¹P/¹H dual-tuned rigid, arc-shaped surface coil (Rapid Biomedical, Germany) was positioned under the right vastus lateralis muscle while the subjects 400 lay prone in a head-first position, shown in FIG. 22. Referring to FIG. 28, an external reference (6 mL plastic vial with an 850 mM concentration of methylenediphosphonic acid (MDP)) was fixed at the center of the coil. MDP was chosen due to its resonance frequency of ˜22 parts per million (ppm) downfield from PCr (which does not overlap any relevant metabolite peaks) (97). In addition, MDP is safe when diluted, is water soluble, and can be placed in a sealed container. After the subject 400 was scanned, a 15 cm diameter, 4 L plastic cylindrical leg phantom containing 35 mM phosphoric acid (H₃PO₄) was placed on the coil and scanned using the same MRS parameters and slab positions so that the data were collected from the same area within the radio frequency excitation field of the coil. The two phantoms are shown in FIG. 28.

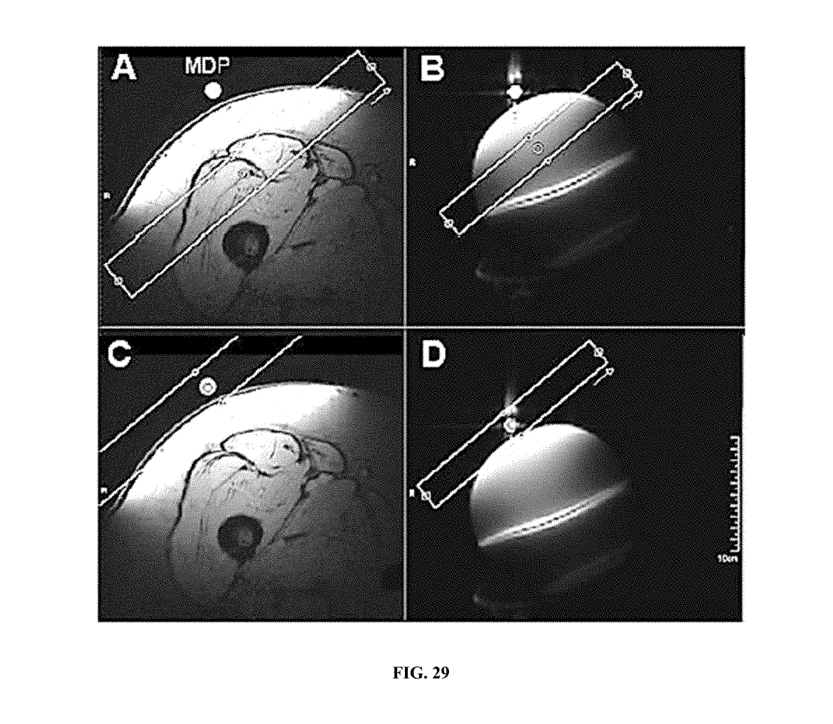

Referring now to FIG. 29, a hydrogen localizer image (35 slices, 200×169×5 mm, TR=498 ms, TE=6.47 ms, BW=120 Hz/pixel) was acquired to visualize the placement of the spectroscopy slabs. A hydrogen spectroscopy voxel (20×20×10 mm, TR=3 s, TE=150 ms, NSA=8, BW=1200 Hz) was placed in the vastus lateralis muscle to find the best shimming parameters for the subject 400. The shimming parameters were manually adjusted until the full width at half maximum (FWHM) of water signal was as low as possible, with a target value of 20 Hz. These same shimming values were then used for the rest of the protocol for the phosphorus spectroscopy sequences. A slice-selective phosphorus MRS slab sequence (TR=10 s, TE=2.3 s, NSA=16, slab thickness 25 mm, BW=3000 Hz) was performed in the quadriceps muscles of the subject 400 and in the leg phantom described above. Using the hydrogen images of the upper right leg, the two dimensional (2D) spectroscopy slab was positioned in the subject 400 to mainly include the vastus lateralis muscle and exclude as much subcutaneous fat and bone as possible. The same position was used when scanning the leg phantom.

The same MRS sequence was used with the slab position centered over the vial of MDP. This external reference spectrum was obtained twice, first with the subject 400 and then again with the leg phantom. These data were used to correct for differences in coil loading. Coil loading is due to the inductive coupling that occurs between the RF surface coil, the conductive tissues in the leg, or the conductive solution in the leg phantom. Since the electrical properties of the subject's leg and the leg phantom are not the same, slight differences in the coil tuning and matching may result. The vial of MDP remained in the same place relative to the coil throughout the experiment; therefore, its spectral peak area did not change. The change in the MDP peak area (which is due to the changes of the coil's RF field) was used to correct for the effect of coil loading on the metabolite peak height. A visual depiction of the positioning of these four scans is presented in FIG. 29.

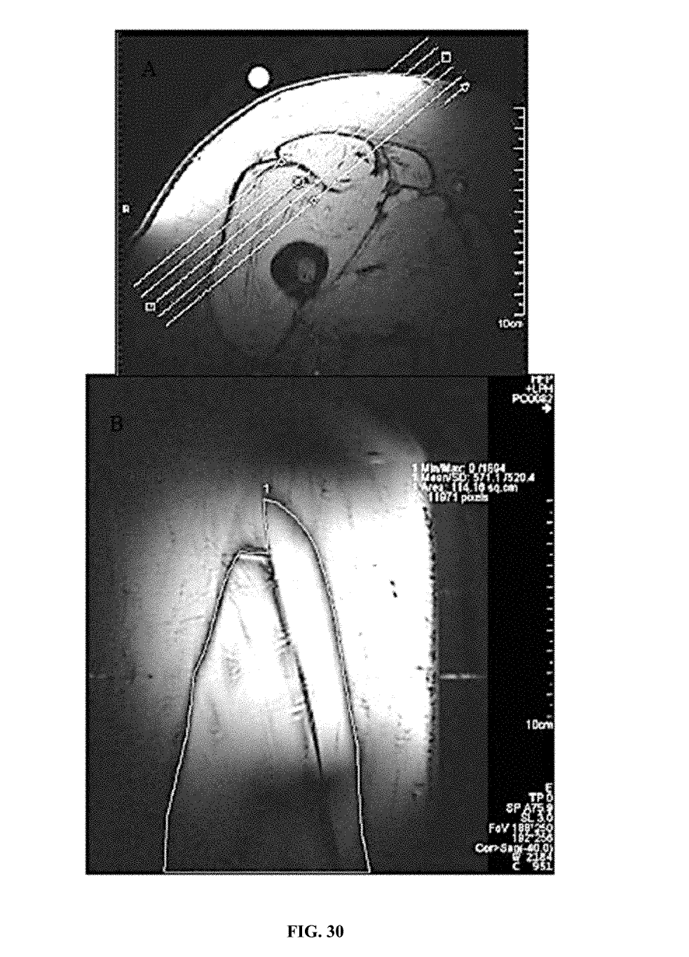

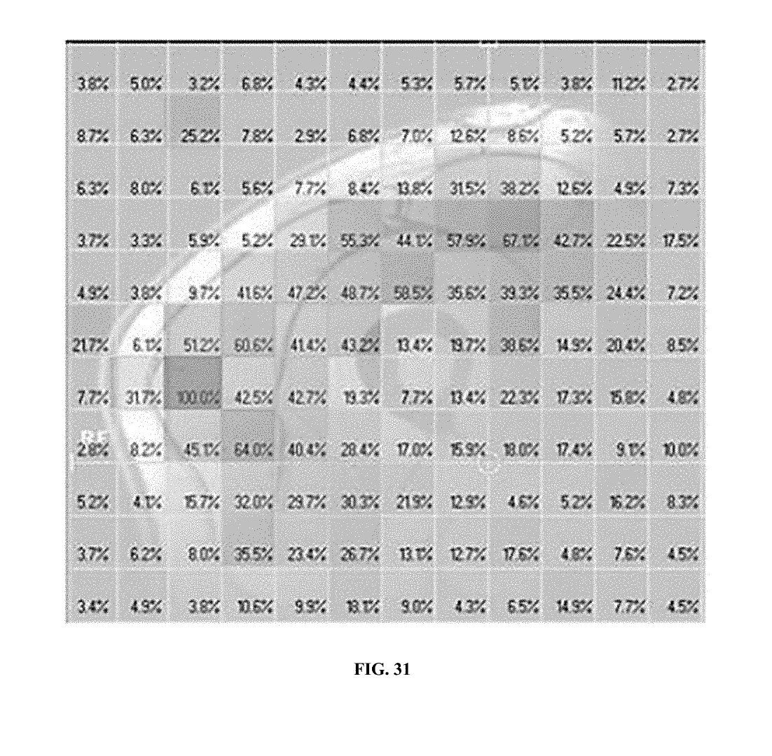

Volume correction was performed using a five-slice×5 mm 1-hydrogen magnetic resonance imaging (¹H-MRI) scan, in the same location as the ³¹P-MRS slab, both in the muscle and the phantom (pictured in FIG. 30) to estimate the volume of muscle tissue in the subject 400, or liquid in the phantom, contained within that 25 mm thick ³¹P-MRS slab. In one subject, a 16×16 matrix, multi-voxel spectroscopic image (TR=3000 ms, TE=2.3 ms, FOV=200 mm, thickness==25 mm, NSA=2) was acquired to validate that the source of the ³¹P signal was exclusively from the muscle (pictured in FIG. 31).

The effective T1 of PCr was measured using the same sequence as the MRS slab with varying TR values (0.5, 1, 3, 6, 10 s) in all 5 healthy volunteers. The T1 values for PCr ranged from 572-893 ms, which verified that the spectroscopy sequence with a TR of ten seconds was sufficiently long that a T1 correction factor did not affect the calculation of the absolute Quantitation.

The absolute concentration of PCr ([PCr]) was determined by the following equation:

[PCr]=[H3PO4]×(A/Ap)×(AMDPref/AMDP)×(Vp/V)

where [H3PO4] is the concentration of phosphoric acid in the leg phantom, A is the amplitude of the metabolite peak in the subject, Ap is the amplitude of the H3PO4 peak from the phantom, AMDPref is the amplitude of the MDP peak with the phantom, AMDP is the amplitude of the MDP peak with the subject, Vp is the volume of the phantom in the slab, and V is the volume of the subject's muscle in the slab.

For comparison, a [PCr]conv also was calculated assuming that [ATP]conv is 8.2 mM:

[PCr]conv=8.2 mM×(APCr/AATP)

where APCr is the amplitude of the PCr peak in the subject and AATP is the amplitude of the ATP peak in the subject.

The molar concentrations of the phosphorus and hydrogen metabolites for the T2DM and NGT subjects are listed in Table 5 while the metabolic findings are in Table 6.

TABLE 5

| PARAMETER | NGT | T2DM | p-value |
|---|---|---|---|
| [PCr] (mM) | 28.6 ± 3.2 | 24.6 ± 2.4 | 0.002* |
| [Pi] (mM) | 2.80 ± 0.57 | 2.79 ± 0.41 | 0.93 |
| [ATP] (mM) | 7.18 ± 0.60 | 6.37 ± 1.05 | 0.02* |
| [PCr]_{conv}¹ (mM) | 32.5 ± 1.7 | 32.2 ± 4.1 | 0.82† |
| pH | 7.00 ± 0.03 | 7.00 ± 0.05 | 0.99 |
| [Cr]² (mM) | 21.8 ± 5.6 | 19.7 ± 2.5 | 0.28 |
| [ADP]² (μM) | 33 ± 9 | 31 ± 6 | 0.45 |
| [ADP]_{conv}³ (μM) | 26 ± 8 | 35 ± 9 | 0.03* |
| [IMCL] (mmol/kg)⁴ | 6.87 ± 1.15 | 8.99 ± 1.46 | 0.004* |

Note.
Data from 11 T2DM subjects and 14 NGT subjects from Table 3-1.
Data are shown as Mean ± Standard Deviation.
¹[PCr]_{conv} uses the ratio of the desired metabolite to the amplitude of ATP, and then multiplying by [ATP] using the typical assumption that [ATP]_{conv} = 8.2 mM.
²2 NGT and 1 T2DM subjects were excluded due to poor data
³[ADP]_{conv} assumes [ATP]_{conv} = 8.2 mM and [PCr] + [Cr] = 42.5 mM
⁴Subset of 9 NGT and 8 T2DM subjects
*p < 0.05
†Unequal variance from Levene test

TABLE 6

| PARAMETER | NGT | T2DM | p-value |
|---|---|---|---|
| Fasting plasma glucose (mg/dl) | 93 ± 6 | 140 ± 23 | <0.001 |
| HbA1c (%) | 5.5 ± 0.3 | 7.5 ± 0.7 | <0.001 |
| Matsuda Index | 4.76 ± 2.75 | 1.80 ± 1.18 | 0.003 |
| Disposition Index | 5.91 ± 2.95 | 0.66 ± 0.71 | <0.001† |
| From E-H Clamp¹ and Biopsy² | | | |
| M | 8.37 ± 2.07 | 4.39 ± 2.17 | <0.001 |
| M/I | 4.03 ± 1.52 | 1.77 ± 0.68 | <0.001 |
| Mitochondrial Density (mito/μm²) | 0.19 ± 0.04 | 0.19 ± 0.02 | 0.96 |

Note.
Data from 14 NGT and 11 T2DM subjects from Table 3-1.
Data are presented as Mean ± Standard Deviation
¹Subset of 12 NGT and 9 T2DM participated in the E-H clamp
²8 NGT and 6 T2DM subjects
†Unequal variance from Levene test

There was a significant difference in the [PCr] between NGT (28.6±3.2 mM) and T2DM (24.6±2.4 mM) groups (p=0.002), as well as [ATP] (NGT: r=7.18±0.60 and T2DM: r=6.37±1.05, p=0.02). There were minimal differences in the means for [Pi], pH, [Cr] and [ADP] between T2DM and NGT subjects, when our calculated metabolites are used to find a more precise [ADP]. Using the conventional method, which assumes a constant and uniform [ATP] of 8.2 mM for all subjects, the difference in the baseline [PCr] was minimally discernable between the groups (p=0.8). For all 23 subjects the [Pi] was 2.80±0.50 mM.

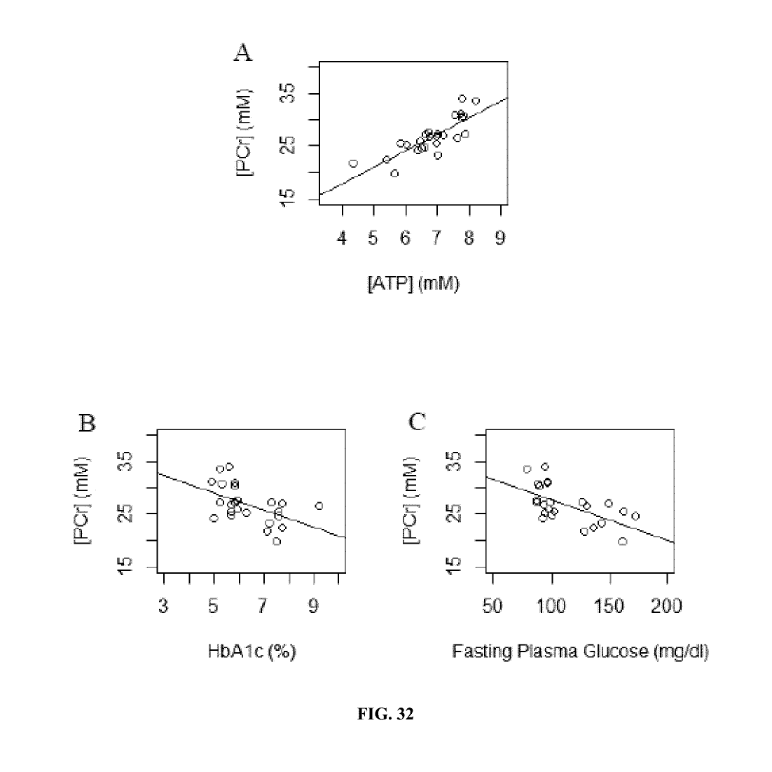

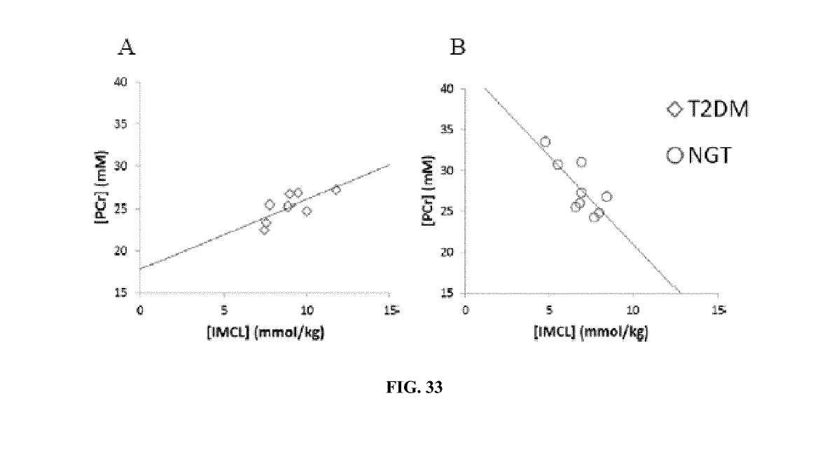

A subset of 12 NGT and 11 T2DM subjects had 1H-MRS spectra of sufficient quality to calculate muscle creatine concentrations [Cr] which produced nonsignificant differences of 21.8±5.6 mM and 19.7±2.5 mM in the NGT and T2DM groups, respectively (p=0.28). When [ADP] is calculated using the absolute values for baseline metabolites, minimal difference is found between groups (p=0.45). With the assumptions used in the conventional method of calculating [ADP], a false significant difference between groups is shown with the T2DM subjects exhibiting a higher ADP. Referring now to FIG. 32, the mean values for [ADP], 33±9 μM in NGT subjects and 31±6 μM in T2DM subjects, were not significant (p=0.45). [ATP] and [PCr] were not significantly correlated with age in either group, but were highly correlated with each other in all subjects (r=0.82, p=<0.001). [PCr] was significantly correlated with HbA1c (ρ=−0.51, p=0.01) and FPG (ρ=−0.63, p=<0.001). Scatter plots of [PCr] versus HbA1c and Fasting Plasma Glucose (FPG) are depicted in FIG. 32. [IMCL] correlated significantly with the Matsuda Index (p=0.009), Disposition Index (p=0.006), FPG (p=0.03), HbA1c (p=0.02), and M/I (p=0.03) in all subjects and was significantly different between groups (p=0.004). Interestingly, referring to FIG. 33, [IMCL] correlated negatively with [PCr] in the NGT group while it correlated positively with [PCr] in the T2DM group. The subset of individuals with [IMCL] measurements were well matched for age (p=0.92) and BMI (p=0.82).

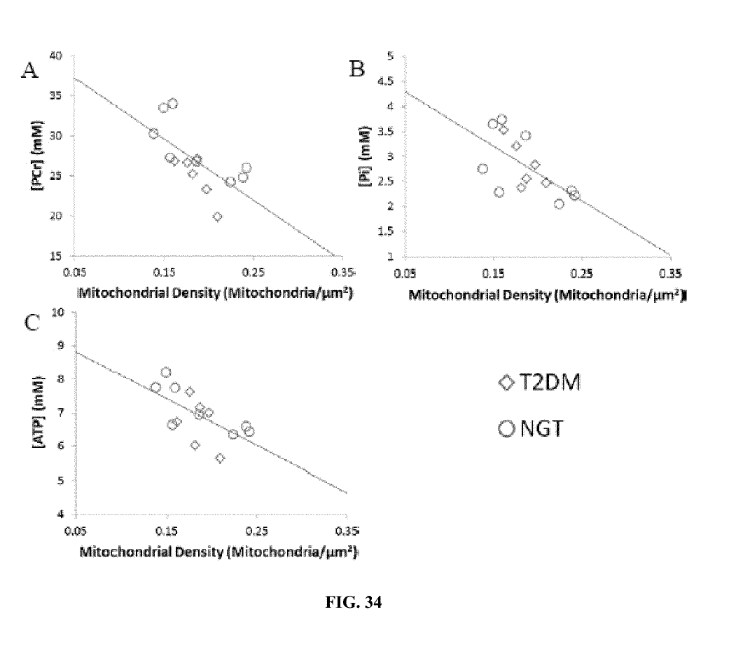

The mitochondrial number density evaluated using transmission electron microscopy (TEM) was able to be performed in 8 NGT and 6 T2DM subjects and no statistically significant difference between groups was found (p=0.96). However, mitochondrial density did correlate with the [PCr] in all subjects (r=−0.67, p=0.009), referring to FIG. 34. The baseline concentrations of Pi and ATP show similar negative relationships with mitochondrial density in all subjects (r=−0.61, p=0.02 and r=−0.63, p=0.02).

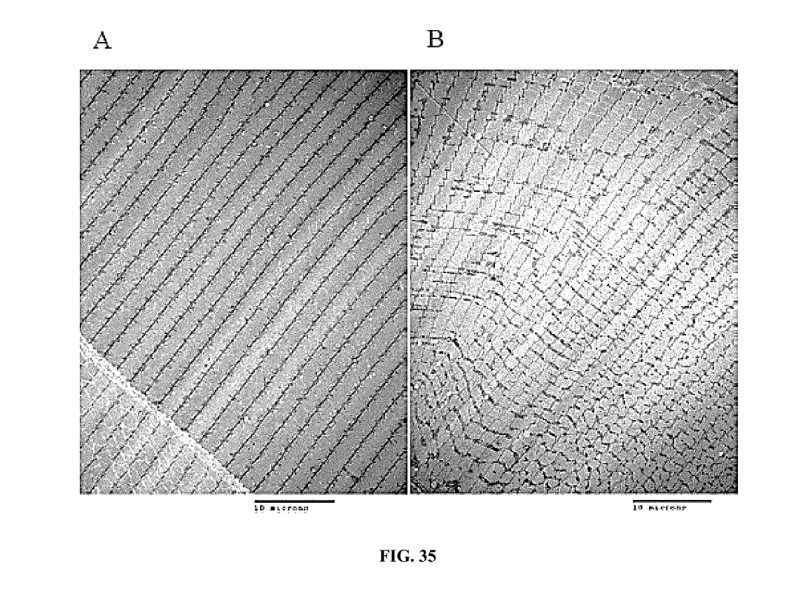

Referring to FIG. 35, another finding from the TEM images was a loss of muscle structure and regularity in the T2DM subjects. Instead of the mitochondria being structured in neat pairs along the z line, T2DM subjects have areas with more mitochondria and less.

These studies demonstrate differences in skeletal muscle metabolism between T2DM and NGT subjects at rest. The [PCr] and [ATP] were significantly reduced in T2DM subjects, while [Pi] was similar in both groups. However, not to be bound by any theory, the strong correlation between [PCr] and both HbA1c and FPG support the concept that reduced baseline skeletal muscle [PCr] is associated with poor glycemic control, not insulin resistance. Mitochondrial density correlated negatively with the resting phosphorus metabolites [PCr], [Pi], and [ATP], indicating that individuals with a greater quantity of mitochondria may have lower cytosolic phosphorus levels. Minimal significant correlation with age or BMI for [PCr] or [ATP] was found. In these studies, minimal difference was found in mitochondrial number density between the two groups. It did correlate strongly and negatively with [PCr] across all subjects as well as in each group individually. However, since a highly significant difference was found between the [PCr] levels in the two groups, the baseline level of PCr may be affected by more than just an individual's mitochondrial density. Similar negative correlations of mitochondrial density with [Pi] and [ATP] were also found in all subjects. Furthermore, it was observed that T2DM muscle has a loss of structure and mitochondria placement uniformity compared to NGT subjects. Without being bound to any theory, this could be due to the previously described shift of T2DM mitochondria from intramuscular to subsarcolemmal locations.

While designing a repeatable exercise-based MRS protocol, it was readily apparent subjects would have different physical abilities and exercise techniques. It was presumed the amount of effort a subject put forth would not only vary between people, but may vary on a single subject from day-to-day.

The goal was to fatigue the VL m. in a manner that lowered the PCr level to 50% of baseline within the five minute exercise period. To determine the relative amount of effort a subject 400 exerted, the apparatus 100 was instrumented with a device to record the force the distal leg/foot applied to the actuating bar 110. A second device to measure the position of the actuating bar 110 at 20 ms intervals was designed to determine the velocity (meters/second) based on the change of displacement from one sample to the next.

After the apparatus 100 was built and thoroughly tested, the force applied versus voltage output of the strain gauge 520 was characterized. Also the position versus elastic restoring force or resistance of the elastomeric band was determined. The sampled data was used to determine the energy, in Joules or calories, burned during the exercise period. Joules can also be described as Newton-meters or kg*m²/s². The kinematic data processing is described in detail in the following section.

The data processor calculates the proportional work using three different methods simultaneously with a variance of ±3.5% and a maximum variance between any two methods of about 7.5%, in Joules. The exact number of exercise seconds is known and the exact amount of force in Newton (or kg) is also known from the calibration tests. Referring to FIG. 19, it can be seen that the area under the force line (the graphical image with respect to CH0) can be accurately summated if each 20 ms data point is treated as a volume of force rather than a scalar of force. The exact number of Newton-seconds

$$( kg force * \frac{m}{s} )$$

can be determined, which again is proportional to the energy expended. A Newton-second is also called the “impulse”, which adds energy to a mechanical system.

After discovering a suitable cement, the entire circuit 530 was assembled in a day. 30 gauge lead wires were kept to a minimum length so one half of the Wheatstone bridge 532 was not out of balance. With the correct placement of the strain gauge 520 device in the bridge, the circuit 530 may maintain balance. The circuit 530 has behaved properly since it was first powered up. It is very sensitive even when only a small force is applied. When stationary, the instrument is so sensitive that the DAQ 514 records the acoustical sound of certain MR sequences.

During characterization of the force applied versus the amplifier 510 output, it was found the gain could be lowered substantially. During subject testing, several subjects applied so much force that the instrumentation amplifier 510 would rail against the supply voltage. This event appeared as a flat top in the wave form where the strain signal was clipped. No useful data could be obtained above Vss. A gain of 500 was settled upon with a V_{ref}=0.5 volts. This produced a full range of values during exercise.

V_{ref} is produced by a low cost op-amp configured as a voltage follower from a 1K ohm potentiometer. The op-amp provides a low impedance source for V_{ref} input of the AD8223 instrumentation amplifier 510 as recommended by the manufacturer.

Integrating the USB-201 DAQ was straight forward. A Windows7 laptop found an appropriate USB device driver when the USB cable was plugged in. A green LED on the module indicated an active interface.

A run once program was required to locate the DAQ hardware and add the correct configuration parameters for the DAQ being used. The National Instruments Measurement and Automation Explorer (NI MAX) software was installed automatically from the software and drivers disk that shipped with the DAQ. Once NI MAX detected the USB-201, channels, tasks and interfaces could be created.

Signal connections were made by capturing bare conductors under tiny screw terminals. Each analog signal required its own ground connection.

Two appropriate software programs to visualize exercise activity and capture the numerical were found on the MCC website. Both programs could emulate a strip recorder and record about 16,000 data points. Both DAQami and TracerDAQ were used to acquire, view and log the analog data connections on three of eight available channels.

Still referring to FIG. 19, Channel 0 502 is used to acquire the strain information. Channel 1 504 is used to set the instrumentation amplifier 510 reference voltage after the subject 400 has been placed in the bore 150. In practice, V_{ref} has been left at 0.5 volts. Channel 2 506 obtains information indicating the position of the articulating arm over its duty cycle. TracerDAQ was mostly used in the experiments presented here for the sake of simplicity.

The data collection file is automatically named with a timestamp and saved when the acquisition ends. The operator can then use the program to convert the history files (.sch) to a comma delimited spreadsheet file (.csv) or a text file (.txt). The .csv file is used to quantify the amount of work performed by the subject 400 during exercise.

The data processor was adapted to accept the data format of these two programs and eliminated the original plan to write a data capture program in LabView.

It could be said any value above the base line of channel 0 502 is work being performance by the subject 400. With a time domain of 50 Hz on the x-axis, a data point is the force applied at 20 ms intervals. The area under the curve is the summation of force every 20 ms. The resistance (i.e. weight) of the actuating bar 110 to movement has been measured and referenced by two different methods.

Using an electronic scale that has been calibrated for earth's gravity (g=9.81 m/s²) the tension or number of kilograms of force (kg*m/s²) exerted by the elastomeric band was measured for every 1.25 cm (0.5 inch) of displacement (s) of the actuating bar 110 over the full range of motion (0-37.5 cm, 0-14.7 inches). Alternately, a measurement of force vs the output voltage of the strain gauge 520 was taken. Data were tabulated and entered into the data processor spreadsheet as lookup tables.

The elastomer can be regarded as a spring following Hooke's law providing a restoring force or weight (W). Using an ordinary scale calibrated for kilograms, the equivalent mass (kg) or more correctly, mass-force of the elastomer was characterized over the duty cycle of the actuating bar 110.

With the known mass, it is simple to convert to Newton (kg m/s²) which is a unit of force by:

N=kg*9.81 m/s²

On earth (a=g=9.81 m/s²) 1 kg equals 9.8 Newton.

If one Newton displaces an object by one meter (a Newton meter), the amount of energy expended is obtained.

$$1 N * 1 m = 1 \frac{kg * {m}^{2}}{{s}^{2}} ,$$

which is equal to 1 Joule (J).

The first method to find work uses the kinetic energy formula. The data from the position indicator provides the exact location of the actuating arm in its arc of movement. Taking the delta between two adjacent samples provides the amount of displacement (meters) during that 20 ms interval. Multiplying the displacement by 50 Hz gives the instantaneous velocity (v) in m/s.

Knowing the mass (kg), the displacement (m) and the sample time (s), the kinetic energy equation is used to find the energy expended during the sample period:

$$E = \frac{1}{2} m {v}^{2} ( kg * \frac{{m}^{2}}{{s}^{2}} ) Joules ( J )$$

Using this method any negative displacement, shown as negative velocity is nullified by squaring ν. At a moment when ν=0, an estimated energy value is used based on the current applied force and the current displacement of the actuating bar 110. Once the energy expended during each 20 ms period has been calculated, all 16,000 data points (5 minutes) are summed, yielding the total energy imparted to the apparatus 100 by a subject 400 in joules. The conversion to watts (J/s) or calories or horsepower or BTUs is a simple conversion.

The second method to determine energy imparted to the apparatus 100 uses the concept of impulse. Known is the amount of force a subject 400 is putting on the actuating bar 110 every 20 ms. Since the period is small, one could presume for one period of time a constant amount of force is applied. Therefore, the Newton-second is:

$$N * t ( sec ) = \frac{kg * m}{{s}^{2}} * s = kg * \frac{m}{s}$$

which is also known as the impulse.

The summation of the amount of force applied by the subject per sample time is then multiplied by the maximum displacement of each leg extension. This method produces the largest variation compared to the other two techniques, but it is still between 3.5% and 7.5% of the other two methods.

The third method uses the classical mechanics definition of work.

$$Work ( Joules ) = F ( Newton ) * S ( meters ) or ( kg \frac{m}{{s}^{2}} * m )$$

For each data sample the strain gauge 520 output is converted into Newton and is multiplied by the tiny displacement during each 20 ms period to calculate joules per period. The summation of all 16,000 samples is the total number of joules imparted to the apparatus 100. The exact displacement is known by the position indicator instrument being sampled simultaneously with the strain measurements. A partial arc equation using the radius and the position in radians is used to find the displacement S.

A few reasonable adjustments had to be made using this method such as using the absolute value of a negative displacement since the VL m. was still providing a resistive force. Also at the point where the actuating bar 110 reached its fully depressed position, the delta displacement equaled zero as did the instantaneous velocity. But in this case the maximum force was being applied to the bar. To compensate for this condition, the impulse value is used to estimate work during this time interval.

Incidentally, a Newton-meter is also used as a unit of torque (T) where one Newton of force is applied to the end of a one meter cantilevered bar. However, even though torque and work look dimensionally equal, they are not the same thing. But, multiplying torque by a dimensionless displacement, such as circular radians, does equate to work in joules.

These calculations only provide a quantity of energy proportional to the amount of chemical energy actually consumed by the VL m. The calculations are accurate for all subjects though, making them useful for comparison of the efforts exerted.

TABLE 7
Energy and Force Imparted to the Exercise Apparatus

| Subject # | | Total Force | Total Work (J) | Total Energy (J) | N-meters (J) |
|---|---|---|---|---|---|
| Age/Sex | Date | (Newton-Sec) | (W = F * S) | (KE = 1/2 mv²) | (KE = rads * τ) |
| #1 30/M | 4/15 | 15578 | 4833 | 4832 | 4835 |
| #1 30/M | 4/29 | 4380 | 3220 | 8842 | 2219 |
| #1 30/M | 5/6 | 4192 | 2128 | 2688 | 1966 |
| #1 30/M | 5/29 | 12135 | 3899 | 8316 | 2895 |
| #1 30/M | 9/26 | 18422 | 5934 | 6494 | 6770 |
| #2 27/M | 4/17 | 28127 | 5022 | 6077 | 4813 |
| #2 27/M | 4/25 | 9462 | 3281 | 8348 | 1886 |
| #2 27/M | 6/12 | ND | ND | ND | ND |
| #3 39/M | 4/18 | 28840 | 9271 | 11235 | 9069 |
| #3 39/M | 4/25 | 33551 | 7365 | 7522 | 8512 |
| #3 39/M | 5/20 | 28391 | 7022 | 8905 | 8343 |
| #3 39/M | 6/5 | ND | ND | ND | ND |
| #4 56/M | 5/5 | 12282 | 3365 | 4815 | 3250 |
| #4 56/M | 5/8 | 5542 | 4449 | 7227 | 3857 |
| #4 56/M | 5/23 | 19653 | 5111 | 5429 | 4983 |
| #5 16/M | 7/14 | 23629 | 6424 | 6793 | 7657 |
| #5 16/M | 7/15 | 24062 | 6710 | 7931 | 7457 |
| #6 31/F | 5/8 | 32225 | 7163 | 7334 | 8065 |
| #6 31/F | 6/18 | 25115 | 4723 | 4321 | 5626 |
| #7 30/F | 5/8 | 35064 | 7762 | 7714 | 7880 |
| #7 30/F | 5/16 | 26406 | ND | ND | ND |
| #8 65/F | 5/22 | 21126 | 4009 | 4318 | ND |
| #8 65/F | 5/26 | 34671 | ND | ND | ND |
| #9 31/F | 8/1 | 39128 | 10224 | 11966 | 9860 |
| #9 31/F | 8/6 | 28812 | 8830 | 10075 | 8951 |
| #10 59/M | 4/29 | 22652 | 5286 | 6716 | 4990 |
| #10 59/M | 5/5 | 27116 | 7455 | 9302 | 6786 |
| #10 59/M | 5/8 | 17028 | 4291 | 5637 | 3962 |
| #10 59/M | 5/16 | 34146 | 6432 | 6588 | 6354 |
| #10 59/M | 5/16 | 45751 | 9095 | 10110 | 8841 |
| #10 59/M | 5/29 | 25070 | 3210 | 3060 | 3655 |
| #10 59/M | 6/5 | 19672 | 1016 | 1000 | 2192 |
| #10 59/M | 6/16 | 29336 | 6834 | 7130 | 6985 |
| #10 59/M | 7/8 | 10981 | 4718 | 5903 | 4776 |
| #10 59/M | 8/11 | 14995 | 3190 | 3550 | 3632 |
| #10 59/M | 9/30 | 16680 | 4714 | 5438 | 4428 |
| #11 25/F | 5/6 | 28205 | 5206 | 7151 | 6628 |
| #11 25/F | 5/29 | 31003 | 6957 | 9260 | 5954 |
| #11 25/F | 6/11 | 26120 | 8024 | 9148 | ND |
| #11 25/F | 6/16 | 26592 | 6230 | 7002 | 5857 |
| #11 25/F | 7/23 | ND | ND | ND | ND |
| #11 25/F | 7/24 | 21530 | 6521 | 8229 | 6217 |
| #11 25/F | 8/12 | 15204 | 4853 | 6987 | 4691 |

Data quality improved as the exercise apparatus 100 and data processor evolved.

The above description and the accompanying drawings provide an example of the technical idea of the present disclosure for illustrative purposes only. Those having ordinary knowledge in the technical field, to which the present disclosure pertains, will appreciate that various modifications and changes in form, such as combination, separation, substitution, and change of a configuration, are possible without departing from the essential features of the present disclosure. Therefore, the embodiments disclosed in the present disclosure are intended to illustrate the scope of the technical idea of the present disclosure, and the scope of the present disclosure is not limited by the embodiment. The scope of the present disclosure shall be construed on the basis of the accompanying claims in such a manner that all of the technical ideas included within the scope equivalent to the claims belong to the present disclosure.

Various other configurations of the components described above are also envisioned and the description herein should not be read to limit the embodiments of the disclosure.

Although the disclosure has been described with respect to certain features, it should be understood that the features and embodiments of the features can be combined with other features and embodiments of those features.

Although the disclosure has been described in detail, it should be understood that various changes, substitutions, and alterations can be made hereupon without departing from the principle and scope of the disclosure. Accordingly, the scope of the present disclosure should be determined by the following claims and their appropriate legal equivalents.

The singular forms “a,” “an,” and “the” include plural referents, unless the context clearly dictates otherwise.

Optional of optionally means that the subsequently described event or circumstances can or may not occur. The description includes instances where the event or circumstance occurs and instances where it does not occur.

Ranges may be expressed throughout as from about one particular value, and to about another particular value. When such a range is expressed, it is to be understood that another embodiment is from the one particular value and to the other particular value, along with all combinations within said range.

As used throughout the disclosure and in the appended claims, the words “comprise,” “has,” and “include” and all grammatical variations thereof are each intended to have an open, non-limiting meaning that does not exclude additional elements or steps.

As used throughout the disclosure, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that that the mere use of the term “first” and “second” does not require that there be any “third” component, although that possibility is contemplated under the scope of the present disclosure.

While the disclosure has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present disclosure may suitably comprise, consist or consist essentially of the elements disclosed and may be practiced in the absence of an element not disclosed.

What is claimed is:

1. An exercise apparatus for measuring work performed by a subject in a magnetic resonance imaging device, comprising:
an actuating bar,
an elastomer band positioned inside the actuating bar,
a tower and roller bearing, and
a pivot base supporting the actuating bar, the elastomer band positioned inside the actuating bar, and the tower and roller bearing.

2. The apparatus according to claim 1, further comprising at least four attach points to position, mount, and secure the apparatus to a secondary device.

3. The apparatus according to claim 2, wherein the secondary device is a magnetic resonance imaging device.

4. The apparatus of claim 3, wherein the apparatus further comprises a magnetic resonance coil.

5. The apparatus according to claim 4, wherein the magnetic resonance coil is positioned close to an isocenter of a magnet of the magnetic resonance imaging device, and the vastus lateralis muscle of the subject and a primary axis of the magnetic resonance coil are parallel to a magnetic field within a bore of the magnetic resonance imaging device.

6. The apparatus according to claim 1, wherein a material is selected from a group consisting of polyvinylchloride (PVC), common PVC cement, and non-magnetic stainless steel, nylon, and combinations thereof.

7. The apparatus according to claim 1, wherein the apparatus is adjustable to accommodate subjects from 60 inches to 77 inches in height.

8. The apparatus according to claim 1, wherein the actuating bar has a single adjustment point to extend length of the actuating bar to match a subject's distal leg length.

9. The apparatus according to claim 1, wherein the apparatus provides for the correct positioning of a leg of the subject.

10. The apparatus according to claim 9, wherein the apparatus isolates movement of the leg in the y-axis direction.

11. The apparatus according to claim 1, wherein the actuating bar comprises a pivot point with a slip bearing to withstand torque in two axes.

12. A method for executing a magnetic resonance imaging protocol comprising:
(a) positioning and attaching an apparatus to a scanner couch of a magnetic resonance imaging device; and
(b) positioning a subject on the apparatus, wherein the apparatus comprises:
a surface coil positioned close to an isocenter of a magnet of the magnetic resonance imaging device,
an actuating bar,
an elastomer band positioned inside the actuating bar,
a tower and roller bearing,
a pivot base supporting the actuating bar, the elastomer band positioned inside the actuating bar, and the tower and roller bearing,
at least four attach points to position, mount, and secure the apparatus to the magnetic resonance imaging device; and
a coil holder comprising the surface coil cradled in the coil holder and secured by a hook and loop strips.

13. A force-sensing instrument for use in a magnetic resonance imaging device, comprising a strain gauge cemented to a surface of an actuating device that is operable in a magnetic resonance imaging device.

14. The force-sensing instrument according to claim 13, wherein the strain gauge is a copper foil electrical circuit, which is bonded to a thin plastic film.

15. The force-sensing instrument according to claim 13, wherein the strain gauge comprises two strain devices cemented to an actuating bar of the actuating device, and separated by about 180±2 degrees.

16. The force-sensing instrument according to claim 15, wherein the two strain devices form a Wheatstone bridge circuit along with two precision resistors, such that the two strain devices are positioned to sense strain in only one axis from a torque in a bar thereof.

17. The force-sensing instrument according to claim 13, further comprising a position indicator device.

18. The force-sensing instrument according to claim 13, further wherein the force-sensing instrument fits within an actuating bar of the actuating device.

19. A magnetic resonance imaging device comprising the force-sensing instrument according to claim 13, wherein the actuating device is adjustable to accommodate subjects from 60 inches to 77 inches in height.

20. A method of using the force-sensing instrumentation of claim 13, comprising the steps of:
attaching the force-sensing instrumentation to an ergometer attached to the scanner table of a magnetic resonance imaging device;
adjusting the force-sensing instrumentation to accommodate a subject;
operating the force-sensing instrumentation by the subject;
delivering data obtained by the force-sensing instrumentation to a control room;
performing a magnetic resonance imaging scan of the subject;
delivering data obtained from the magnetic resonance imaging scan of the subject to the control room;
further wherein the step of operating the force-sensing instrumentation and the step of performing a magnetic resonance imaging scan overlap.

21. The method of claim 20, further comprising the steps of:
determining a relative amount of work being done by the subject while operating the force sensing instrumentation based on the data obtained by the force-sensing instrumentation and the data obtained by the magnetic resonance imaging scan.

22. The method of claim 20, further wherein data obtained by the force-sensing instrumentation is delivered to the control room using Cath RJ45 connector jacks.

23. The method of claim 20, further wherein data obtained by the force-sensing instrumentation is delivered to the control room using a Cat7 cable.

24. The method of claim 20, further wherein the force-sensing instrumentation comprises:
a low noise circuit that is operable while in the presence of time varying magnetic fields produced during the performing a magnetic resonance imaging scan.
